(19) United States
(12) Patent Application Publication
(10) Pub. No.: US 2022/0054839 A1
(43) Pub. Date: Feb. 24, 2022

(54) S1 NERVE ROOT STIMULATION SYSTEMS AND METHODS

(71) Applicant: Kenneth Chapman, New York, NY (US)

(72) Inventor: Kenneth Chapman, New York, NY (US)

(21) Appl. No.: 17/405,573

(22) Filed: Aug. 18, 2021

Related U.S. Application Data
(60) Provisional application No. 63/067,003, filed on Aug. 18, 2020.

(51) Int. Cl. A61N 1/36; A61N 1/05

(52) U.S. Cl. CPC A61N 1/36071; A61N 1/36157; A61N 1/0558; A61N 1/36062

(57) ABSTRACT

Systems and methods for stimulating a spinal nerve root of an S1 nerve of a patient involve advancing a treatment device lateral to a thecal sac, through a spinal canal, and into and through an S1 foramen of a sacral vertebral body of the patient. A distal portion of an electrode region is disposed anterior to an anterior border of the sacral vertebral body. Electrical stimulation is delivered from the electrode region to a treatment location disposed on a spinal nerve root of an S1 nerve, distal to a dorsal root ganglion of the S1 nerve, distal to a dorsal ramus of the S1 nerve, and adjacent to an entry zone of a gray rami communicans of the S1 nerve. Delivery of electrical stimulation modulates visceral afferent fibers and postganglionic sympathetic neurons at the treatment location. Treatments can be provided to patients presenting with peripheral vascular disease and other conditions.

### CROSS-REFERENCE

This application claims the benefit of U.S. Provisional Patent Application No. 63/067,003 filed Aug. 18, 2020, the disclosure of which is incorporated herein by reference.

### BACKGROUND

Peripheral vascular disease (PVD) refers to a disorder of the circulatory system that impedes blood flow to a region of the body, leading to poor oxygenation of the tissue or ischemia. High blood pressure, diabetes, high cholesterol, smoking, and autoimmune disorders are among the more common causes of PVD. Forms of PVD include chronic venous insufficiency, deep vein thrombosis (DVT), thrombophlebitis, and varicose veins. Frequently, PVD patients develop severe pain and disability in their lower extremities due to poor circulation. Peripheral arterial disease (PAD) is a form of PVD that occurs within the arteries, and affects 10-15% of the population and about 20% of people over the age of 60. Typically, vascular surgeons are the managers of patients with PVD.

Medical management, revascularization through angioplasty, bypass surgery, and ultimately amputation fall within the treatment algorithm for PVD. Although these treatment modalities can provide relief to patients presenting with PVD, still further improvements are desired. Embodiments of the present invention address at least some of these outstanding needs.

### SUMMARY

Devices and methods configured for use in modulating the afferent visceral (sensory) input to preganglionic (premotor) sympathetic neurons and the postganglionic (motor) sympathetic neurons at the proximal spinal nerve root (S1) can produce neuromodulation effects that reach far beyond the spinal nerve being stimulated, to adequately cover an entire distal lower extremity. Because PVD can significantly affect the feet and distal lower extremities (e.g. due to their greater distance from the heart and the presence of smaller diameter vessels), embodiments of the present invention are well suited for use in treating patients who are at risk of developing PVD or who present with PVD.

In one aspect, embodiments of the present invention encompass methods of treating a patient presenting with peripheral vascular disease. Exemplary methods can include advancing a treatment device along an insertion path into the patient, where the treatment device includes an electrode region, a spacer region located proximal to the electrode region, and a tined region located proximal to the spacer region, and where the insertion path is disposed lateral to a thecal sac, through a spinal canal, and into and through an S1 foramen of a sacral vertebral body of the patient. Methods can also include anchoring the treatment device within the patient, such that the tined region is disposed dorsal to the spinal canal and dorsal to a posterior border of the sacral vertebral body of the patient, the spacer region is disposed within the spinal canal and lateral to the thecal sac, a proximal portion of the electrode region is disposed within the spinal canal, and a distal portion of the electrode region is disposed anterior to an anterior border of the sacral vertebral body of the patient. Methods can further include delivering an electrical stimulation treatment from the electrode region of the treatment device to a treatment location of the patient, where the treatment location is disposed on a spinal nerve root of an S1 nerve of the patient, distal to a dorsal root ganglion of the S1 nerve, distal to a dorsal ramus of the S1 nerve, and adjacent to an entry zone of a gray rami communicans of the S1 nerve, and where delivery of the electrical stimulation treatment operates to simultaneously modulate visceral afferent fibers and postganglionic sympathetic neurons at the treatment location. As discussed elsewhere herein, some methods may involve placing an electrical stimulation implant lead at the sacral plexus (e.g. through a sacral foramen onto the nerve root at the sacral plexus). In some cases, electrical contacts or electrodes of the implant can be positioned anterior (outside) to the spinal canal. In some cases, electrical contacts or electrodes of the implant can be positioned at the anterior border of the sacral vertebral body. In some cases, electrical contacts or electrodes of the implant can be disposed dorsal to the anterior border of the sacral vertebral body (e.g. within the spinal canal). In some cases, electrical stimulation can be delivered to a desired treatment location on the S1 nerve root and the stimulation or electrical signal spreads throughout certain nerve fiber pathways of the sacral plexus. In some cases, the step of anchoring the treatment device within the patient includes embedding one or more tines of the tined region of the treatment device in thoracolumbar fascia tissue of the patient. In some cases, the spacer region of the treatment device has a length of about 20 mm In some cases, the spacer region of the treatment device has a length with a value within a range from about 22 mm to about 24 mm In some cases, the spacer region of the treatment device has a length with a value within a range from about 15 mm to about 25 mm According to some embodiments, the step of delivering the electrical stimulation treatment includes delivering electrical energy with a frequency having a value within a range from about 0.5 Hz to about 20 Hz. In some embodiments, the step of delivering the electrical stimulation treatment includes delivering electrical energy with a frequency having a value that is greater than 20 Hz. In some embodiments, the step of delivering the electrical stimulation treatment includes delivering electrical energy with a frequency having a value that is greater than 100 Hz. In some embodiments, the step of delivering the electrical stimulation treatment includes delivering electrical energy at a first current value and subsequently at a second current value, where the first current value and the second current value are separated by a current increment, and where the current increment has a value within a range from about 0.005 mA to about 0.025 mA. In some embodiments, the step of delivering the electrical stimulation treatment includes delivering electrical energy at a constant current.

In another aspect, embodiments of the present invention encompass systems for treating a patient presenting with peripheral vascular disease or diabetic peripheral neuropathy. Exemplary systems can include a treatment device having an electrode region, a spacer region located proximal to the electrode region, and a tined region located proximal to the spacer region. The treatment device can configured to be anchored within the patient, such that the tined region is disposed dorsal to an S1 posterior sacral foramen of the patient, the spacer region is disposed within the spinal canal and lateral to a thecal sac of the patient, a proximal portion of the electrode region is disposed within the spinal canal, a distal portion of the electrode region is disposed anterior to an S1 anterior sacral foramen corresponding to the S1 posterior sacral foramen, and an electrode of the electrode region is disposed at a spinal nerve root of an S1 nerve of the patient, distal to a dorsal root ganglion of the S1 nerve, distal to a dorsal ramus of the S1 nerve, and adjacent to an entry zone of a gray rami communicans of the S1 nerve. Exemplary systems can also include a processor, an electronic storage location operatively coupled with the processor and processor executable code stored on the electronic storage location and embodied in a tangible non-transitory computer readable medium. The processor executable code, when executed by the processor, can cause the treatment device to deliver an electrical stimulation treatment from the electrode region of the treatment device to the patient, to simultaneously modulate visceral afferent fibers and postganglionic sympathetic neurons of the patient. In some cases, the spacer region of the treatment device has a length of about 20 mm In some cases, the spacer region of the treatment device has a length with a value within a range from about 22 mm to about 24 mm In some cases, the spacer region of the treatment device has a length with a value within a range from about 15 mm to about 25 mm In some cases, the electrical stimulation treatment includes electrical energy with a frequency having a value within a range from about 0.5 Hz to about 20 Hz. In some cases, the electrical stimulation treatment includes electrical energy with a frequency having a value that is greater than 20 Hz. In some cases, the electrical stimulation treatment includes electrical energy with a frequency having a value that is greater than 100 Hz. In some cases, the electrical stimulation treatment includes electrical energy provided at a first current value and subsequently at a second current value, where the first current value and the second current value are separated by a current increment, and where the current increment has a value within a range from about 0.005 mA to about 0.025 mA. In some cases, the electrical stimulation treatment includes electrical energy provided at a constant current.

In yet another aspect, embodiments of the present invention include systems and methods for stimulating a spinal nerve root of an S1 nerve of a patient. Exemplary methods may include advancing a treatment device along an insertion path into the patient, where the treatment device includes an electrode region, a spacer region located proximal to the electrode region, and a tined region located proximal to the spacer region, and where the insertion path is disposed lateral to a thecal sac, through a spinal canal, and into and through an S1 foramen of a sacral vertebral body of the patient. Exemplary methods may include anchoring the treatment device within the patient, such that the tined region is disposed dorsal to the spinal canal, the spacer region is disposed lateral to the thecal sac, and an electrode of the electrode region is disposed to an anterior border of the sacral vertebral body of the patient. Exemplary methods may also include delivering an electrical stimulation treatment from the electrode of the treatment device to a treatment location of the patient, where the treatment location is disposed on a spinal nerve root of an S1 nerve of the patient, distal to a dorsal root ganglion of the S1 nerve, distal to a dorsal ramus of the S1 nerve, and adjacent to an entry zone of a gray rami communicans of the S1 nerve, and where delivery of the electrical stimulation treatment operates to simultaneously modulate visceral afferent fibers and postganglionic sympathetic neurons at the treatment location. In some cases, a patient may be selected for receiving the treatment because they present with a condition such as peripheral vascular disease, diabetic peripheral neuropathy, complex regional pain syndrome, or Raynaud's syndrome.

In still another aspect, exemplary methods can include advancing a treatment device along an insertion path into the patient, where the treatment device includes an electrode region, a spacer region located proximal to the electrode region, and a tined region located proximal to the spacer region, and where the insertion path is disposed lateral to a thecal sac, through a spinal canal, and into and through an S1 foramen of a sacral vertebral body of the patient.

In another aspect, embodiments of the present invention encompass methods of treating a patient that include advancing a treatment device along an insertion path into the patient, where the treatment device includes an electrode region, and where the insertion path is disposed through a foramen of a vertebral body of the patient, anchoring the treatment device within the patient, such that a distal portion of the electrode region is in an extraforaminal region, and delivering an electrical stimulation treatment from the electrode region of the treatment device to a treatment location of the patient, where the treatment location is disposed on a spinal nerve root of a spinal nerve of the patient, and where delivery of the electrical stimulation treatment operates to simultaneously modulate visceral afferent fibers and postganglionic sympathetic neurons at the treatment location.

### BRIEF DESCRIPTION OF THE DRAWINGS

The novel features of the invention are set forth with particularity in the appended claims. A better understanding of the features and advantages of the provided system and methods will be obtained by reference to the following detailed description that sets forth illustrative embodiments, in which the principles of the invention are utilized, and the accompanying drawings of which:

FIG. 1 illustrates aspects of S1 spinal nerve root treatment systems and methods, according to embodiments of the present invention;

FIGS. 2A, 2B, and 2C illustrate aspects of S1 spinal nerve root treatment systems and methods, according to embodiments of the present invention;

FIGS. 3A and 3B illustrate aspects of S1 spinal nerve root treatment systems and methods, according to embodiments of the present invention;

FIGS. 3C to 3G depict aspects of nerve treatment systems and methods, according to embodiments of the present invention;

FIG. 4 illustrates aspects of S1 spinal nerve root treatment systems and methods, according to embodiments of the present invention;

FIG. 5 illustrates aspects of S1 spinal nerve root treatment systems and methods, according to embodiments of the present invention;

FIG. 6 illustrates aspects of S1 spinal nerve root treatment methods, according to embodiments of the present invention; and

FIG. 7 illustrates aspects of S1 spinal nerve root treatment methods, according to embodiments of the present invention.

### DETAILED DESCRIPTION OF THE INVENTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Embodiments of the present invention are well suited for use by vascular surgeons, as they are the managers of patients with PVD. Exemplary embodiments encompass stimulating the S1 nerve root using certain parameters, at a certain location in the spine, to improve distal limb perfusion. According to some embodiments, placing a treatment electrode or lead at the desired S1 nerve root treatment location can be facilitated with fluoroscopic guidance, using an X-ray machine that vascular surgeons routinely use for vascular interventions such as angioplasty. The desired treatment outcome can include increases in blood flow and tissue perfusion with concomitant pain relief secondary to modulation of sensory afferent input and reduced ischemia.

According to some embodiments, an exemplary location to selectively modulate the autonomic nerve fibers is at the proximal spinal nerve root adjacent to the entry zone of the gray rami communicans. Given the convergence of nerve fibers within both the spinal cord and within the sympathetic chain, targeting the S1 sacral nerve root alone provides a superior means of stimulation for sympathetic innervation of the lower extremities. Such techniques can be performed in patients with PVD, instead of using spinal cord stimulation (SCS) and other treatment modalities.

Peripheral vascular disease (PVD) refers to a disorder of the circulatory system that impedes blood flow to a region of the body, leading to poor oxygenation of the tissue or ischemia. High blood pressure, diabetes, high cholesterol, smoking, and autoimmune disorders are among the more common causes of PVD. Frequently, patients develop severe pain and disability in their lower extremities due to poor circulation. Despite correcting the underlying causes with conservative medical treatment, PVD may not improve in advanced cases, and common treatments include revascularization through angioplasty or bypass surgery. However, these interventions may not be enough to restore adequate blood flow/perfusion to the extremities, leading to ulcers, gangrene, and possible amputation. Embodiments of the present invention provide a solution to address such shortcomings of these existing treatments.

Embodiments of the present invention provide advantages over other known PVD treatments such as spinal cord stimulation (SCS), because SCS only stimulates one type of sensory nerve fiber in the spinal cord, limiting the effects it can have on other types of nerves. In contrast, embodiments of the present invention encompass the selective stimulation or modulation of both the afferent (sensory) visceral input to preganglionic (premotor) sympathetic neurons and the postganglionic (motor) sympathetic neurons within the spinal nerve root (S1), which allows the neuromodulation effects to reach far beyond the spinal nerve being stimulated to adequately cover the entire distal lower extremity.

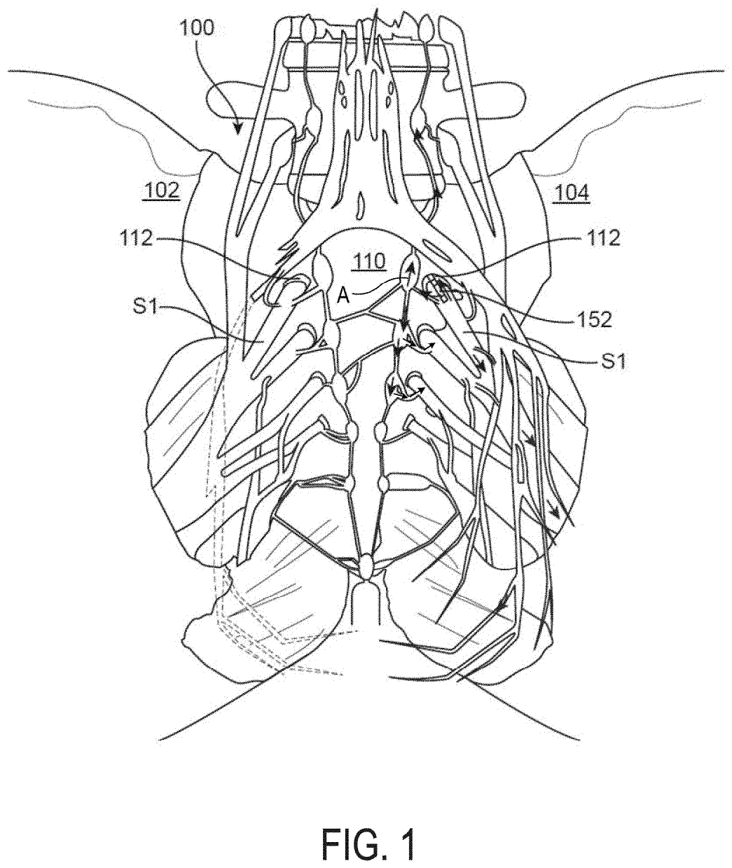

Turning now to the drawings, FIG. 1 illustrates aspects of a treatment method and device, according to embodiments of the present invention. The sacrum 100 is a triangular assembly of fused sacral vertebrae (typically five, e.g. S1-S5) located at the base of the spine. The sacrum 100 provides support for the spine, accommodates the spinal nerves, and articulates with the iliac bones 102, 104. In some cases, the sacrum 100 may be referred to as a monolithic structure composed of multiple (e.g. 5) fused sacral vertebrae. In some cases, the sacrum 100 may be referred to as a sacral vertebral body. As shown in FIG. 1, the sacrum 100 includes a pelvic or anterior surface 110, is dorsally convex, and is positioned in a wedge-like manner between the two iliac bones 102, 104.

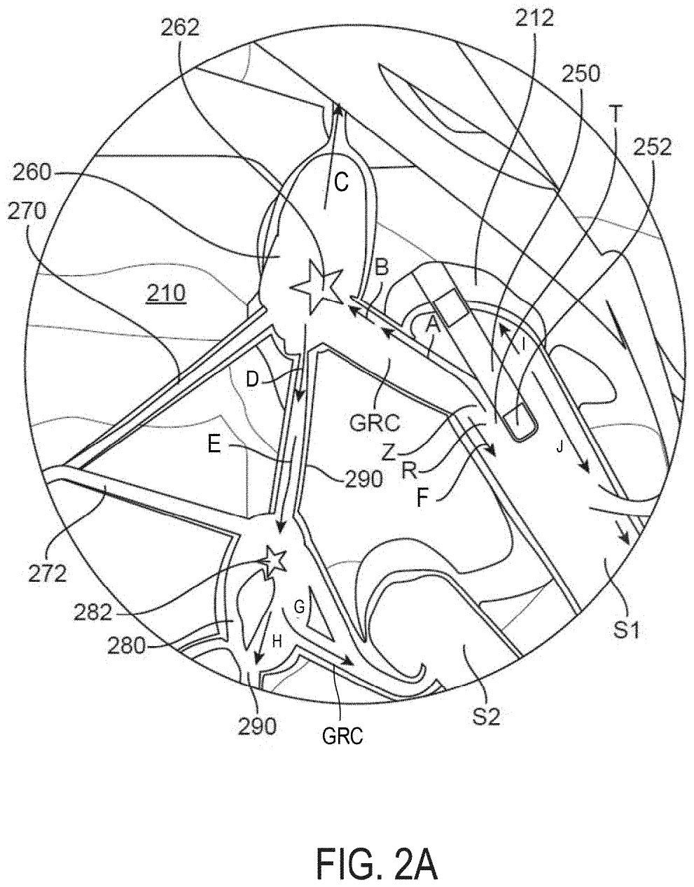

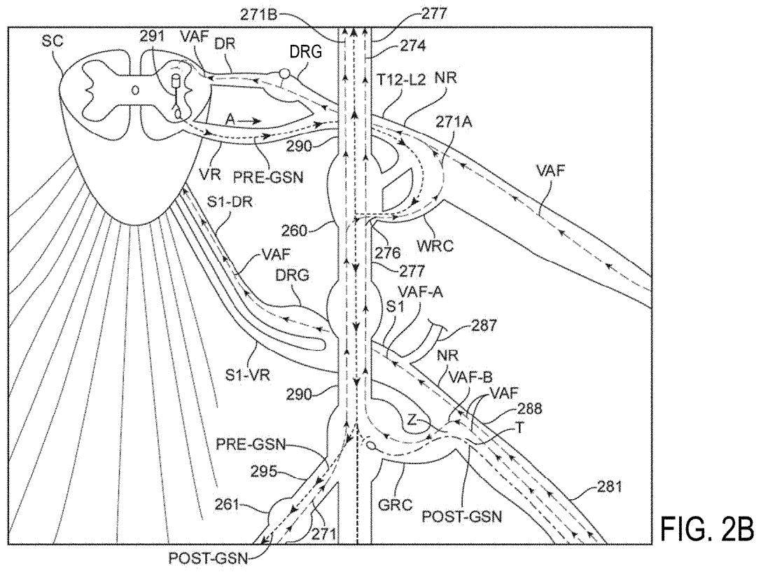

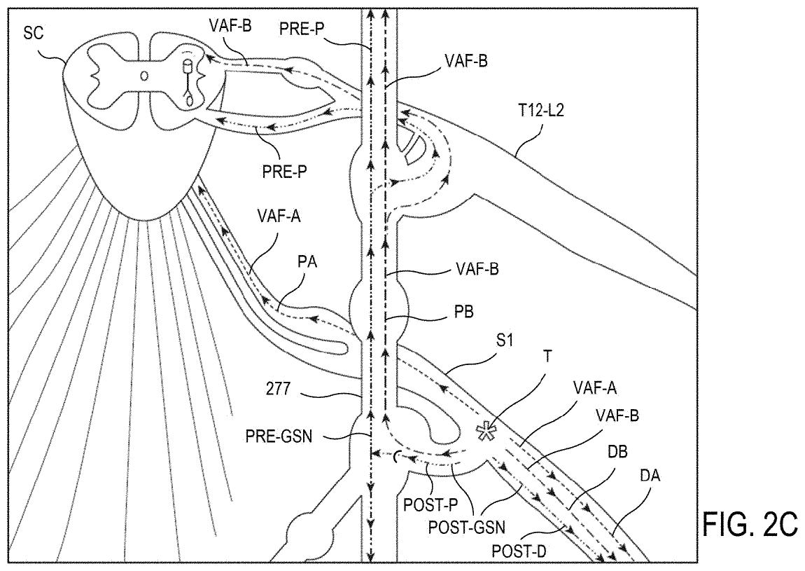

The sacral nerves are pairs of spinal nerves which exit the sacrum 100 at the lower end of the vertebral column. For example, as shown here, on the anterior concave surface 110, there can be four pairs of anterior sacral foramina that allow passage of the anterior rami of the sacral nerves. The anterior sacral foramina are typically larger than the corresponding posterior sacral foramina (not shown here). The anterior divisions of the sacral nerves exit anteriorly through the anterior foramina Hence, for example, the S1 sacral nerve (S1) exits anteriorly through the S1 anterior sacral foramen 112. As shown here, a distal portion 152 of an electrode region of a treatment device extends anteriorly from the S1 anterior (pelvic) sacral foramen 112. When electrical stimulation is delivered to a desired treatment location on the S1 nerve root near the gray rami communicans, the stimulation or electrical signal spreads throughout certain nerve fiber pathways of the sacral plexus, for example as indicated by the electrical signal fiber stimulation pathway arrows, one of which is labeled, for clarity, as arrow A. Additional details regarding certain electrical signal fiber stimulation pathways are described elsewhere throughout this disclosure, for example with reference to FIG. 2A and FIG. 2C.

FIG. 2A illustrates aspects of a treatment method and device, according to embodiments of the present invention. As shown here, a distal portion 252 of an electrode region of a treatment device extends anteriorly through an anterior (pelvic) S1 sacral foramen 212, and is disposed anterior to an anterior border or anterior (pelvic) surface 210 of a sacral vertebral body of a patient. An exemplary treatment method can include delivering an electrical stimulation treatment from the electrode region 250 of the treatment device to a treatment location T of the patient. In some embodiments, the treatment location T is disposed on a spinal nerve root R of an S1 nerve of the patient, distal to a dorsal root ganglion (not shown here) of the S1 nerve, and adjacent to an entry zone Z of a gray rami communicans GRC of the S1 nerve.

When electrical stimulation is administered or delivered to a nerve root, an electrical charge is provided to an area on the nerve, resulting in altered neural activity. The stimulation can operate to lightly depolarize the nerve, and thus produce an action potential that is propagated along the axon in both directions. In some cases, this may result in modulation of existing sensory input that occurs in the nerve.

As shown in FIG. 2A, an electrical signal can travel from the treatment location T on the S1 spinal nerve root R, through the gray rami communicans GRC (as depicted by arrows A and B), to the paravertebral ganglion 260, which is part of the sympathetic chain. The sympathetic chain runs up and down the spinal cord from the cervical spine to the coccyx. Sympathetic fibers run through the sympathetic chain system. Arrow C depicts the spread of electrical stimulation from the paravertebral sympathetic ganglion 260 up the sympathetic chain, after the stimulation has traveled through synapse 262. Such stimulation is implicated in the treatment of PVD. This represents the spread of flow of the electrical signals (e.g. action potential propagation) from the spinal cord preganglionic sympathetic (premotor) neuron within the sympathetic chain. Arrows A, B, and C correlate to upward flow of stimulation along the visceral afferents. Arrow F represents the flow of stimulation from the electrode lead along the postganglionic fibers that are stimulated to travel down the peripheral nerve toward the blood vessels. Further details regarding the neural fiber pathways and signal flow directions are provided herein with reference to FIG. 2B.

For additional anatomical context, FIG. 2A depicts a splanchnic nerve 270, where a postganglionic fiber can travel to an internal organ or tissue, for example in the abdominal/pelvic cavity. Similarly, splanchnic nerve 272 operates as a nerve branch (communicating nerve) that carries visceral afferent and sympathetic efferent fibers to prevertebral ganglia or midline structures like blood vessels, bladder, and the like.

According to some embodiments, splanchnic nerves are paired visceral nerves (nerves that contribute to the innervation of the internal organs), and carry fibers of the autonomic nervous system (visceral efferent fibers) as well as sensory fibers from the organs (visceral afferent fibers). In some cases, the splanchnic nerve 270 operates as a nerve branch (communicating nerve) that carries visceral afferent and sympathetic efferent fibers to prevertebral ganglia or midline structures like blood vessels, bladder, and the like.

As depicted by arrows D and E, an electrical signal can descend from a superior paravertebral ganglion 260 to an inferior paravertebral ganglion 280 along the sympathetic chain 290. According to some embodiments, the sympathetic chain provides fibers that connect connecting paravertebral ganglia on either side of vertebral column For example, as shown here, the sympathetic chain provides fibers that connect connecting paravertebral ganglia (e.g. ganglion 260, ganglion 280) on one side of vertebral column. As further discussed herein with reference to FIG. 2B, sympathetic preganglionic axons can travel from any level of spinal cord origin (T1-L2) in ascending or descending direction to synapse on different postganglionic neurons in paravertebral ganglia (or prevertebral ganglia) at distant spinal levels.

The paravertebral ganglion 280 provides another paravertebral ganglion site where preganglionic axons synapse onto postganglionic neuron. As shown here, within the sympathetic chain, a synapse 282 can occur within the paravertebral ganglion 280, for example a synapse of a sympathetic preganglionic nerve to a sympathetic postganglionic nerve. The postganglionic nerve can leave the ganglion 280 and enter the adjacent spinal nerve R through the gray rami communicans GRC (as depicted by arrow G) and travel down the S2 spinal nerve. Hence, stimulation electrical signals can travel from the sympathetic chain back out to peripheral nerves at other levels such as S2. Stimulation electrical signals traveling along S2 are implicated in the treatment of PVD. The gray rami communicans GRC is a fiber that connects the sympathetic ganglion/chain to the adjacent spinal nerve root where sympathetic postganglionic axons join the spinal nerve (e.g. S2).

As depicted by arrow H, an electrical signal can descend from a superior paravertebral ganglion 280 to an inferior paravertebral ganglion (not shown) along the sympathetic chain 290. According to some embodiments, the sympathetic chain provides fibers that connect connecting paravertebral ganglia (e.g. ganglion 280, and a ganglion inferior to ganglion 280) on either side of vertebral column. Sympathetic preganglionic axons can travel from any level of spinal cord origin (T1-L2) in ascending or descending direction to synapse on different postganglionic neurons in paravertebral ganglia (or prevertebral ganglia) at distant spinal levels. As depicted by arrow I, an electrical signal can ascend upward along the S1 nerve, and as depicted by arrow J, an electrical signal can descent downward along the S1 nerve. Additional aspects of such electrical signal pathways are discussed herein with reference to FIG. 2C.

As shown in FIG. 2B, typically, an electrical signal flows along sympathetic preganglionic axon or fiber as it travels from the spinal cord SC through ventral nerve root VR (e.g. as depicted by arrow A) to a spinal nerve (e.g. any one of thoracic spinal nerve T12, lumbar spinal nerve L1, or lumbar spinal nerve L2), and then exits the spinal nerve through the white rami communicans WRC to enter the sympathetic ganglia/chain 290, for example at paravertebral sympathetic ganglion 260. The spinal cord ends distally at the L2 level and becomes the cauda equina, which is a bundle of lumbar and sacral nerves.

As further shown in FIG. 2B, delivery of an electrical stimulation treatment to the treatment location T can operate to modulate visceral afferent fibers VAF (sensory/afferent) and postganglionic sympathetic neurons POST-GSN at the treatment location T. Hence, during a stimulation procedure or treatment, an electrode region of a treatment device can be activated to stimulate a spinal nerve root NR of the S1 nerve, at a treatment location T that is distal to the dorsal root ganglion (DRG) of the S1 nerve. Selective stimulation of the S1 spinal nerve root NR can operate to activate small fibers of the S1 nerve, including sympathetic nerves (e.g. POST-GSN) which can improve blood flow, relieve ischemic pain, and improve limb salvage. Small fibers (here, sensory nerve fibers) also innervate the skin of the extremity and thus stimulation can treat or relieve neuropathic pain.

In FIG. 2B, afferent fibers (e.g. sensory nerves) 271 travel from the viscus or internal organs (not shown), through the prevertebral sympathetic ganglia 261, along a splanchnic nerve 295, to the sympathetic ganglia/chain 290. Thereafter, signals traveling along the afferent fibers can either continue to propagate in a superior direction upward along the chain 290, or they may propagate through the white rami communicans WRC of a spinal nerve (e.g. any one of thoracic spinal nerve T12, lumbar spinal nerve L1, or lumbar spinal nerve L2) to a nerve root of the respective spinal nerve, through the dorsal root DR, and into the spinal cord SC, and thereafter the signals are propagated in a superior direction up the spinal cord along afferent fiber (e.g. toward the brain). In this way, visceral afferent fibers, as part of the autonomic nervous system, can operate to transmit sensory information from the internal organs of the body back to the central nervous system.

Visceral afferent fibers VAF-B travel from the blood vessels in the lower extremities to join the other sensory fibers of the peripheral nerve S1 as it travels back from the lower extremity through the lumbosacral plexus toward the spinal canal. They separate from other visceral afferent fibers VAF-A and somatic (conscious sensation) sensory fibers (not shown) innervating the skin and skeletal muscle that continue to travel up the S1 nerve root to enter the spinal cord at this level and diverge into the gray rami communicans (GRC) to join the visceral afferent fibers 271 of the internal midline organs and tissues coming from the splanchnic nerves (e.g. 295) and travel up the sympathetic chain to reach the target level of the spinal cord where they exit the white rami communicans (WRC) and enter the higher level spinal nerve, pass through the dorsal root ganglion (DRG) where their cell bodies originate, and synapse on the spinal cord at L2 and above to synapse on a spinal cord interneuron 291 that will connect to preganglionic sympathetic neuron to form the autonomic reflex loop.

It can also be seen, that as part of the sympathetic outflow, sympathetic efferent fibers (e.g. motor nerves) travel from the spinal cord SC, and through the ventral root VR as preganglionic sympathetic fibers PRE-GSN (e.g. sympathetic presynaptic fibers). Signals traveling along the preganglionic sympathetic fibers PRE-GSN enter the paravertebral sympathetic ganglia 260, and thereafter can travel in a superior direction along the sympathetic trunk 277 (e.g. toward the cervical sympathetic ganglia), in an inferior direction along the sympathetic trunk 277 (e.g. toward the lumbar sympathetic ganglia), in a first lateral direction toward the prevertebral sympathetic ganglia 261, where the preganglionic sympathetic fibers PRE-GSN synapse with the postganglionic sympathetic fibers POST-GSN, 8and then signals can travel along the postganglionic sympathetic fibers POST-GSN to the viscus (not shown), or in a second lateral direction toward the gray rami communicans GRC, where the preganglionic sympathetic fibers PRE-GSN synapse with the postganglionic sympathetic fibers POST-GSN within the sympathetic ganglion, and then signals can travel along the postganglionic sympathetic fibers POST-GSN, exiting the sympathetic ganglion through the gray rami communicans to enter the spinal nerve and travel in a descending fashion to the blood vessels, sweat glands, and arrector pili muscles (not shown). The postganglionic sympathetic fibers POST-GSN are also sympathetic efferent fibers (e.g. motor nerves). Synapses between preganglionic and postganglionic neurons occur within the sympathetic chain within the paravertebral ganglia (e.g. 260) or prevertebral ganglia (e.g. 261).

In some cases, a viscus can be an internal organ in a main cavity of the body. For example, a viscus can be an intestine in the abdomen. A viscus may also be a kidney, liver, bladder, or sex organ.

As discussed elsewhere herein, the dorsal root ganglion DRG is typically located within the spinal canal, and the treatment location T is located exterior to the spinal canal (e.g. anterior to the anterior border or anterior (pelvic) surface of the sacrum).

As shown in FIG. 2B, the treatment location T is at a location Z that is adjacent to an entry zone Z of a gray rami communicans GRC of the S1 nerve. In exemplary embodiments, the treatment location T may be at or near a ventral ramus 288 of the S1 nerve. In exemplary embodiments, the treatment location T may be distal to a dorsal ramus 287 of the S1 nerve. In exemplary embodiments, the treatment location T is not at the dorsal root ganglion DRG, the dorsal root DR, or the ventral root VR. Typically, the dorsal root DR, the ventral root VR, and the dorsal root ganglion DRG associated with the S1 nerve are located within the body of the sacrum. When following the S1 nerve from the anterior of the sacrum toward the spinal cord SC, it can be seen that as the S1 nerve enters the spinal canal, it splits into the dorsal root S1-DR and the ventral root S1-VR. At the T1-L2 levels, the ventral root is associated with motor function (e.g. contains preganglionic sympathetic fibers) where these neurons originate and exit the spinal cord through the ventral root and split from skeletal motor fibers and diverge into the white rami communicans to enter the sympathetic chain. The dorsal root is associated with sensory function (e.g. contains visceral afferent fibers VAF).

According to some embodiments, sensory nerves, which may also be referred to as visceral (unconscious) afferents or visceral afferent fibers VAF, operate to monitor the physiologic conditions of blood vessels and internal organs, such as blood flow resistance, pH, and temperature to provide input to the sympathetic nervous system to maintain and control the environment. They transmit information to central control centers in the brain such as the hypothalamus and brain stem to determine the whole body response, as well as form direct synapses or connections with sympathetic preganglionic (premotor) neurons in the spinal cord (e.g. via interneuron 291) to initiate rapid reflex responses to local changes.

Innervation patterns for visceral afferent and sympathetic nerves are non-dermatomal, meaning they do not follow the same pattern of innervation of the surface of the body seen with somatic (conscious) innervation. The preganglionic (premotor) sympathetic neurons PRE-GSN originate in the spinal cord (e.g. T1-L2) in between the posterior sensory portion and the anterior motor portion of the cord at the intermediolateral nucleus. They are activated by descending nerve fibers coming from control centers in the brain as well by sensory afferent nerves forming reflex connections in the spinal cord, sometimes through an interneuron 291 that acts as a bridge between the sensory nerve VAF and the sympathetic premotor nerve PRE-GSN. Preganglionic fibers PRE-GSN exit the spinal cord SC through the ventral (motor) nerve root VR into the mixed spinal nerve and enter the sympathetic chain or sympathetic trunk 277 through the white rami communicans WRC (which are present only in the T1-L2 spinal nerves) at the ventral ramus portion of the mixed spinal nerve.

Preganglionic (premotor) sympathetic neurons PRE-GSN synapse in various sympathetic ganglia 260, 261 along the sympathetic chain onto postganglionic sympathetic neurons POST-GSN. In some cases, a preganglionic nerve PRE-GSN can synapse onto multiple postganglionic nerves POST-GSN at a 1:4 to 1:36 ratio that span the entire length of the sympathetic chain. In some cases, a preganglionic nerve PRE-GSN can synapse onto multiple postganglionic nerves POST-GSN at a 1:4 to 1:20 ratio that span the entire length of the sympathetic chain. Therefore, a single preganglionic sympathetic nerve PRE-GSN can amplify its signal by simultaneously transmitting inputs to multiple postganglionic neurons at spinal levels beyond its spinal cord level of origin. The postganglionic (motor) neurons POST-GSN can exit the sympathetic chain 277 through the gray rami communicans GRC (which are present at every spinal level) to join the ventral ramus portion of the spinal nerve at every level of the spinal cord (e.g. ventral ramus 288 of S1 nerve), and then go on to innervate organ tissues including blood vessels, for example via a peripheral nerve 281. Postganglionic fibers can travel down the spinal nerve to reach the target portion of the blood vessel they are innervating, or the fibers can exit the nerve early and travel along the blood vessel walls to reach the distal portions in the lower extremities.

Sensory fibers (e.g. VAF) are involved in a variety of reflexes and feed-forward physiologic processes that control the sympathetic nervous system and these reflexes and processes can be utilized in the treatment of various disorders. Thus, by stimulating sensory fibers in these areas, such as at treatment location T, fundamental reflexes and processes can be affected to lessen the symptoms of a variety of disorders. Fundamentally, stimulating the sensory neuron (e.g. VAF at treatment location T) stimulates an interneuron (e.g. interneuron 291, at the T12-L2 levels) to the sympathetic premotor neuron which acts upon a sympathetic motor neuron via the associated sympathetic ganglion. This inhibits release of norepinephrine, the neurotransmitter used by the sympathetic motor neuron to signal blood vessel constriction, which in turn lessens vascular resistance and improves blood flow to the areas that had suffered from restricted blood flow.

Afferent sensory nerves from the blood vessels and surrounding perfused tissues can make direct reflex synapses or connections with the sympathetic preganglionic (premotor) neurons in the spinal cord to activate rapid responses. In order to do so, they need to ascend from the lower lumbosacral levels (L3-S5) to the higher portion of the spinal cord where the preganglionic neurons reside at the thoracolumbar T1-L2 levels. A majority of the afferent nerves do so by traveling from the blood vessels along the same route the postganglionic nerves take back to the spinal nerve but instead of entering the spinal cord at that level (e.g. VAF-A, via S1), they exit the spinal nerve through the gray rami communicans GRC (e.g. VAF-B, when at the S1 level) to travel along the sympathetic chain 290 and ascend to the appropriate spinal level (L2 or above, in some cases), exit the sympathetic chain through the white rami communicans WRC, travel through the proximal part of the mixed spinal nerve to the dorsal (sensory) root portion and enter the spinal cord, sending branches that synapse directly on preganglionic sympathetic neurons in the interomedialateral nucleus portion of the spinal cord. Targeting these sensory afferent fibers (e.g. at treatment location T) with electrical stimulation can modulate the signals they send, reducing activation of the sympathetic preganglionic (premotor) neurons and therefore reduce vasoconstriction, improving blood flow in diseased vessels where blood flow may be restricted. For example, vessels in the feet and distal lower extremities.

Some ascending afferent fibers may exit the sympathetic chain 290 (e.g. as depicted by fiber path 276) at the lowest white rami communicans level L2 and other ascending afferent fibers may continue to higher levels such as L1 or T12. Different portions of ascending afferent fibers can exit the sympathetic chain at different spinal nerve levels. Typically, these fibers enter the spinal cord at the lowest few levels of the white rami communicans (e.g. T12-L2). It is noted that although only one such fiber path exit point 276 is depicted in FIG. 2B, it is understood that this path could refer to an exit to L2 level, the L1 level, or the T12 level. Hence, where fiber path 276 exits to the L2 level, for example, other ascending afferent fibers 274 can continue extending in a superior direction, for example to exit at the L1 or T12 levels.

It can be seen that afferent fibers (e.g. sensory nerves) 271 traveling from the viscus through the splanchnic nerve 295 and superiorly through the sympathetic chain 290 can take two different paths. For example, some of the ascending afferent fibers 271 can branch to exit the white rami communicans at the T12, L1, and/or L2 level (e.g. 271A), while other ascending afferent fibers (e.g. 271B) can continue up the sympathetic chain to 290 reach higher portions of the spinal cord. In some embodiments, applying stimulation energy at treatment location T would not implicate these afferent fibers 271.

Experimental studies also show that electrical stimulation of isolated sensory fibers from spinal nerves near their central portion can generate action potentials that travel in the opposite direction (away from the spinal cord) toward the periphery where the sensory nerve endings that innervate the blood vessels release molecules referred to as neurotransmitters to induce vasodilation of blood vessels. In this manner, the afferent sensory nerves simultaneously act like efferent (motor) nerves. Hence, applying energy at treatment location T, in addition to sending signals along VAF-A and VAF-B in a proximal direction, also sends signals along VAF-A and VAF-B in a distal direction, to the feet and distal lower extremities, to treat PVD. In this sense, antidromic stimulation back down the sensory nerve will produce vasodilation, and can be referred to as the axon reflex.

An exemplary location to selectively modulate the autonomic nerve fibers and associated sensory nerve fibers is at the spinal nerve root adjacent to the entry zone of the gray rami communicans. PVD typically affects the feet and distal lower extremities more due to the greater distance from the heart and smaller diameter of the vessels. Without being bound by any particular theory, given the convergence of nerve fibers within both the spinal cord and within the sympathetic chain, it is believed that targeting the S1 sacral nerve root alone provides a particularly effective means of stimulation for sympathetic innervation of the lower extremities, for example as compared to SCS.

Modulating the afferent input to the preganglionic sympathetic neurons at the level of the proximal spinal nerve (S1), for example via afferent sensory nerve fibers VAF-A and VAF-B at treatment location T, allows the neuromodulation effects to reach far beyond the S1 spinal nerve being stimulated to adequately cover the entire distal lower extremity. Furthermore, stimulation of postganglionic (motor) sympathetic fibers POST-GSN, for example at treatment location T, can also control the output of these nerves to reduce vasoconstriction in lower extremity blood vessels. Because this modality has the ability to directly modulate small nerve fibers, there are benefits beyond peripheral vascular disease. For example, modulation of the afferent visceral input and the postganglionic sympathetic neurons at a treatment location T on the proximal S1 spinal nerve root NR can be used to treat small fiber neuropathic pain syndromes, instead of using other techniques such as spinal cord stimulation (SCS).

As another example, selective stimulation or modulation at a treatment location T on the proximal S1 spinal nerve root NR of both the afferent (sensory) visceral input to preganglionic (premotor) sympathetic neurons and the postganglionic (motor) sympathetic neurons can be used to treat a patient presenting with diabetic polyneuropathy of the feet. Diabetic peripheral neuropathy (DPN) is a length-dependent axonal polyneuropathy that begins in the distal lower extremities. An estimated 50% of diabetic patients will develop peripheral neuropathy within 25 years after the initial diagnosis of diabetes mellitus. The prevalence of painful diabetic neuropathy (PDN) ranges from 10% to 20% of patients with diabetes and in those with diabetic neuropathy it ranges from 40% to 50%. DPN is characterized by autonomic dysfunction and small vessel arterial disease. As vascular tone is mediated by the sympathetic nervous system (SNS), modulation of the SNS with spinal nerve electrostimulation can induce vasodilation and reduce neuroinflammation through the same mechanisms as outlined above, including disruption of action potential signaling from afferent visceral fibers to block activation of sympathetic nerves.

According to some embodiments, selective stimulation or modulation of both the afferent (sensory) visceral input to preganglionic (premotor) sympathetic neurons and the postganglionic (motor) sympathetic neurons at a treatment location T on the proximal S1 spinal nerve root NR can be used to improve distal limb perfusion.

Because exemplary treatment methods include selectively stimulating or modulating activity of both the postganglionic sympathetic fibers and the visceral afferent fibers (e.g. at the S1 nerve root), such methods can be advantageous over other methods that involve spinal cord stimulation (SCS), where only one type of sensory nerve fiber in the spinal cord is stimulated. Exemplary S1 nerve root stimulation treatments disclosed herein are also distinct from other methods which involve dedicated treatment specifically to S2, S3, or S4 sacral nerve stimulation, which in some cases may be indicated for bladder and bowel control.

With returning reference to FIG. 2B, it is noted that visceral afferent fibers VAF can densely innervate internal organs or viscera 295. The visceral afferent fibers VAF can operate to monitor nociceptive (painful) input. Visceral afferent fibers VAF can also be sensitive to mechanical or chemical stimuli, such as the stretching of heart tissue, blood vessels, and hollow viscera. Visceral afferent fibers VAF can also be sensitive to changes in PCO₂, PO₂, pH, blood glucose, and the temperature of skin and internal organs.

Within the chain 290, preganglionic sympathetic nerve fibers may synapse there and spread potentially in any direction, or they may not synapse there and travel to a ganglion (group of nerves) closer to an end organ. In some cases, a preganglionic neuron may synapse on multiple postganglionic neurons at a ratio of 1:4-1:20 or greater in several ways upon entering the sympathetic chain. In some cases, a preganglionic neuron may synapse on multiple postganglionic neurons at a ratio of 1:4-1:36 or greater in several ways upon entering the sympathetic chain. Axons of presynaptic nerves can terminate in either the paravertebral ganglia (ganglia situated along either side lateral to the vertebral column, e.g. 260) or prevertebral ganglia (ganglia situated midline along the vertebral column, e.g. 261). There are four different paths an axon can take before reaching its terminal. The axon enters the paravertebral ganglion at the level of its originating spinal nerve. After this, it can then either synapse in this ganglion, ascend to a more superior or descend to a more inferior paravertebral ganglion and synapse there, or it can descend to a prevertebral ganglion and synapse there with the postsynaptic cell.

Within the sympathetic chain 290, a synapse can occur within the paravertebral ganglion (e.g. 260), for example a synapse of a sympathetic preganglionic nerve to a sympathetic postganglionic nerve. The postganglionic nerve can leave the ganglion 260 and enter the adjacent spinal nerve through the gray rami communicans GRC and travel down the spinal nerve to target tissue. As further discussed herein with reference to FIG. 2C, stimulation from the electrode to the postganglionic fibers at treatment location T results in stimulation of the postganglionic sympathetic fibers at the point of contact along the electrode which will send stimulation from that point along the spinal nerve in the orthodromic direction down the peripheral nerve toward a target destination (e.g. blood vessels) and will also send stimulation from that point along the spinal nerve in the antidromic direction back up the gray rami to sympathetic chain where stimulation will travel up and down the chain. The gray rami communicans GRC is a fiber that connects the sympathetic ganglion/chain to the adjacent spinal nerve root where sympathetic postganglionic axons join the spinal nerve (e.g. S1).

Stimulation at location T can be provided for the purpose or treatment of relieving neuropathic pain such as neuropathy or complex regional pain syndrome (CRPS) type 1 and 2, for peripheral vascular disease of the lower extremities including peripheral arterial disease, for vasculopathy, and for Raynaud's syndrome of the toes. Stimulation at location T can also be provided for the purpose of improving bowel/bladder control (in some cases, overlapping with S2-S4 stimulation), improving erectile dysfunction, and for the treatment of relieving pelvic pain. Without being bound by any particular theory, it is believed that the effects can be synergistic by directly stimulating the visceral afferents and the postganglionic efferent. If only postganglionic fibers are directly stimulated, the stimulation effects may be limited to the structures the postganglionic neurons reach from S1 peripheral nerve. In contrast, direct stimulation of both the visceral afferents and the postganglionic efferent provides unique access to the sympathetic chain highway and can have far reaching effects by also modulating afferent input to the preganglionic neurons that then disperse throughout. Further, avoiding direct stimulation of the sympathetic preganglionic fibers can reduce unwanted, unintended, and/or unpredictable effects of overstimulation of the sympathetic nervous system. For example, direct stimulation of the sympathetic preganglionic fibers may provide undesirable effects to the chest, mid-abdomen, or may upset other organ functioning.

FIG. 2C illustrates aspects of selective stimulation or modulation of both the afferent (sensory) visceral input to preganglionic (premotor) sympathetic neurons and the postganglionic (motor) sympathetic neurons within the spinal nerve root (S1), which allows the neuromodulation effects to reach far beyond the spinal nerve being stimulated to adequately cover the entire distal lower extremity. For example, when delivering a stimulation to a treatment location T, electrical nerve signals are propagated along the VAF-A and VAF-B visceral afferent pathways in a proximal direction (as reflected by indicia PA and PB, respectively) and are also propagated along the VAF-A and VAF-B visceral afferent pathways in a distal direction (as reflected by indicia DA and DB, respectively). Because visceral afferent signals typically flow toward the spinal cord (e.g. as depicted in FIG. 2B), the delivery of stimulation at treatment location T results in orthodromic flow at PA and PB, and antidromic flow at DA and DB.

Further, when delivering a stimulation to a treatment location T, electrical nerve signals are propagated along the postganglionic (motor) sympathetic fibers POST-GSN in a proximal direction (as reflected by indicia POST-P) and are also propagated along the POST-GSN fiber pathways in a distal direction (as reflected by indicia POST-D). Because postganglionic motor signals typically flow away from the spinal cord (e.g. as depicted in FIG. 2B), the delivery of stimulation at treatment location T results in orthodromic flow at POST-D, and antidromic flow at POST-P. The signals travel along the POST-GSN fibers in a proximal direction, and propagate across a synapse to the preganglionic sympathetic fibers PRE-GSN (e.g. sympathetic presynaptic fibers). Signals traveling along the preganglionic sympathetic fibers PRE-GSN can travel in a superior direction along the sympathetic trunk 277 and in an inferior direction along the sympathetic trunk 277. As shown here, signals can propagate in a proximal, or antidromic flow direction, along PRE-GSN fibers (as reflected by indicia PRE-P).

Hence, stimulation can go in both directions along the S1 nerve, referred to as orthodromic (same direction the nerve normally transmits signals, the natural flow) and antidromic (opposite direction of nerve signaling) and therefore stimulation will travel downstream to the sacral plexus and beyond and also upstream toward the spinal cord.

In other words, stimulation can spread in both directions of the visceral afferent nerves of interest, meaning downstream (antidromic) in the spinal nerve toward the sacral plexus and upstream (orthodromic) up the proximal portion of the spinal nerve towards the spinal cord, and through the gray rami to go up and down the sympathetic chain. The afferent nerves which are stimulated enter through the gray rami to the sympathetic chain, and thereafter those afferent nerves travel upstream, or in a superior direction, along the sympathetic chain. In addition to stimulating visceral afferent nerves, postganglionic sympathetic neurons are also stimulated. These postganglionic sympathetic neurons travel orthodromic down the spinal nerve and antidromic through the gray rami to the sympathetic ganglion and then spread up and down the sympathetic chain. Hence, when stimulation is applied, the electrical signals travel up and down from the point of entry of gray rami.

Preganglionic sympathetic axons at the T1-12 and L1-2 levels travel from the spinal cord through the ventral root (which may be inside of the spinal canal) of the spinal nerve and exit through white rami communicans to enter the adjacent paravertebral ganglia in the sympathetic chain. There are no white rami and preganglionic sympathetic axons traveling at the S1 level. Hence, stimulation of the S1 nerve root does not directly stimulate preganglionic fibers.

As discussed elsewhere herein, experimental studies show that electrical stimulation of isolated sensory fibers from spinal nerves near their central portion can generate action potentials that travel in the opposite direction (away from the spinal cord) toward the periphery where the sensory nerve endings that innervate the blood vessels release molecules referred to as neurotransmitters to induce vasodilation of blood vessels. In this manner, the afferent sensory nerves simultaneously act like efferent (motor) nerves. Hence, applying energy at treatment location T, in addition to sending signals along VAF-A and VAF-B in a proximal direction, also sends signals along VAF-A and VAF-B in a distal direction, to the feet and distal lower extremities, to treat PVD. In this sense, antidromic stimulation back down the sensory nerve will produce vasodilation, and can be referred to as the axon reflex.

Stimulation from the electrode to the postganglionic fibers at treatment location T results in stimulation of the postganglionic sympathetic fibers at the point of contact along the electrode which will send stimulation from that point along the spinal nerve in the orthodromic direction down the peripheral nerve toward a target destination (e.g. blood vessels) and will also send stimulation from that point along the spinal nerve in the antidromic direction back up the gray rami to sympathetic chain where stimulation will travel up and down the chain. The gray rami communicans GRC is a fiber that connects the sympathetic ganglion/chain to the adjacent spinal nerve root where sympathetic postganglionic axons join the spinal nerve (e.g. S1). Exemplary methods involve delivering stimulation energy at treatment location T, so as to directly activate postganglionic sympathetic neurons, and direct stimulation of preganglionic neurons does not occur.

An indirect effect of directly stimulating VAF fibers, is that the modulated visceral afferent signal eventually synapse on to the interneuron connecting it to a preganglionic sympathetic neuron higher up in the spinal cord (e.g. see FIG. 2B), subsequently leading to reduced preganglionic neuron signaling where this neuron travels.

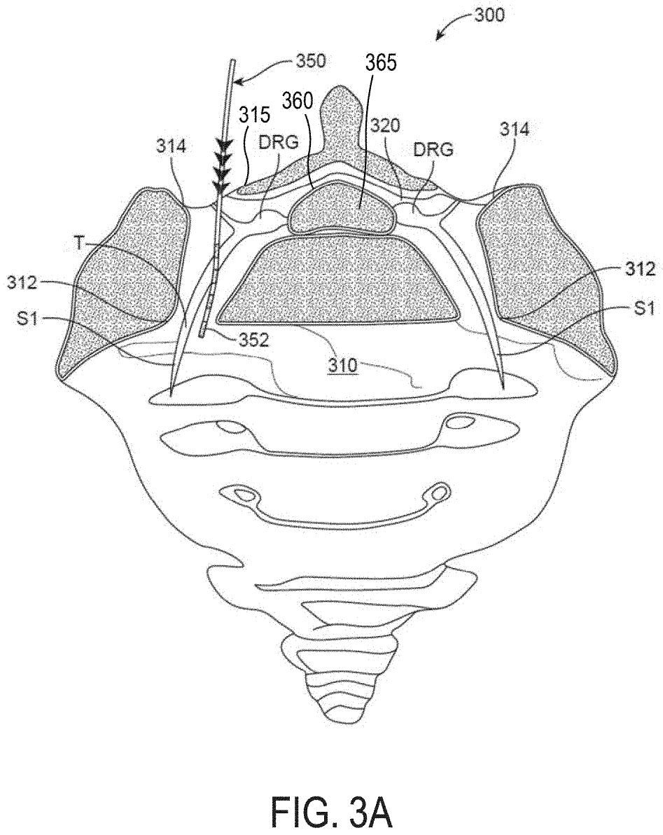

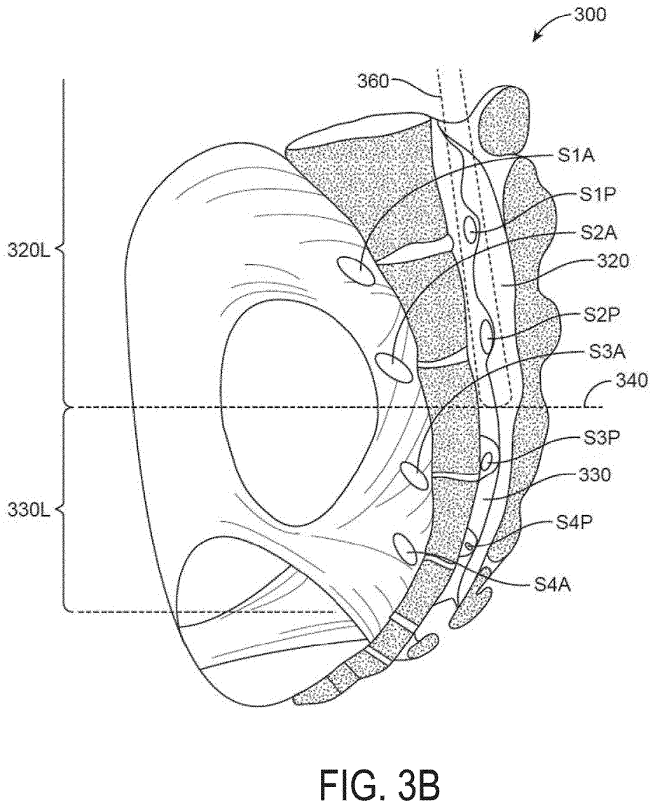

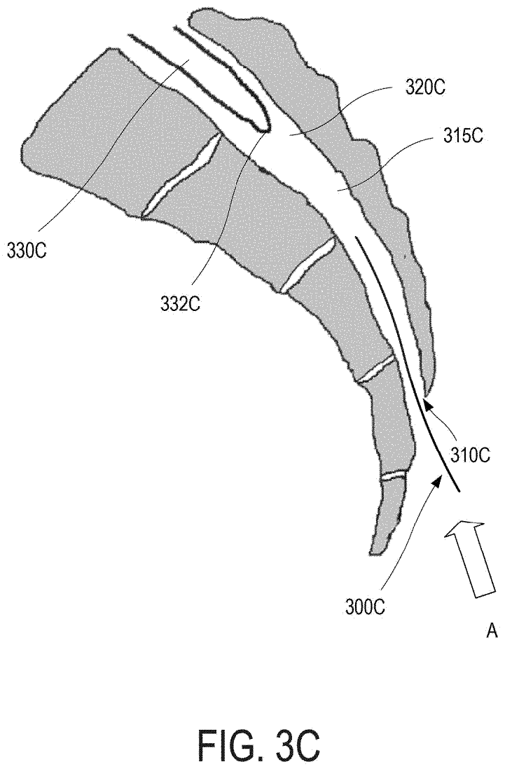

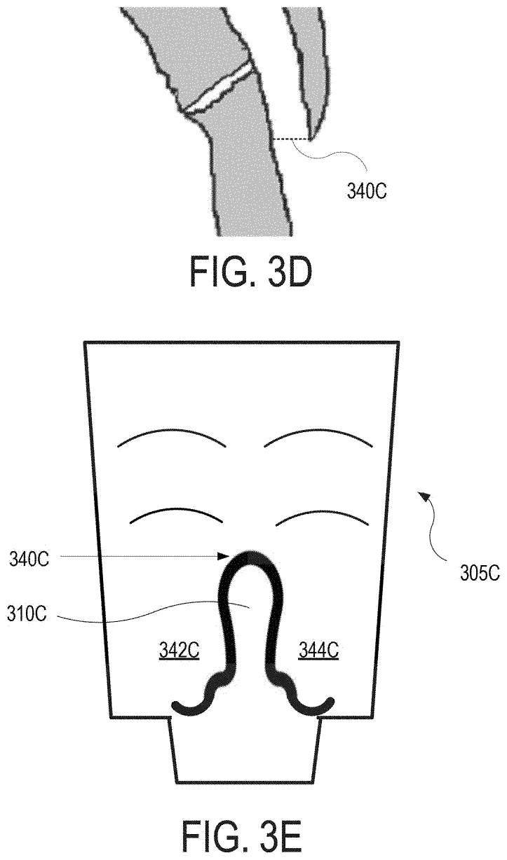

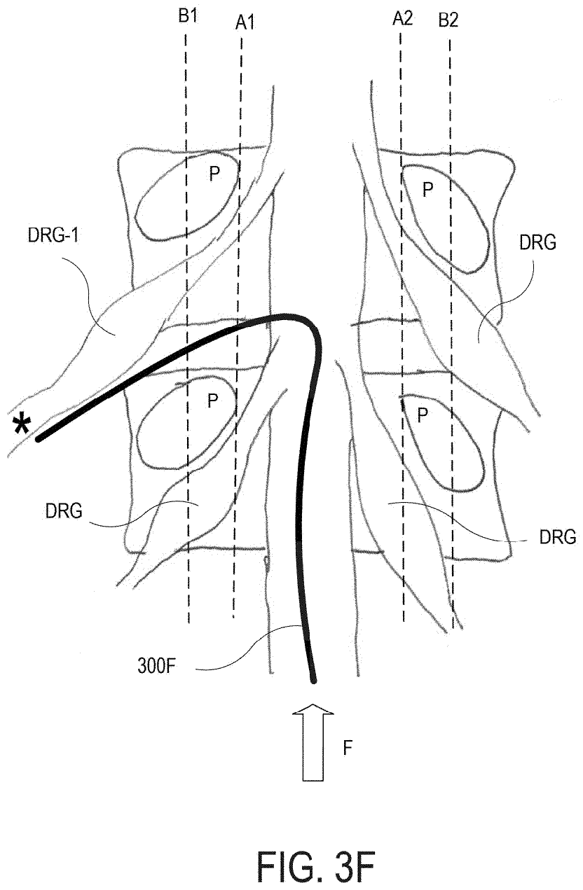

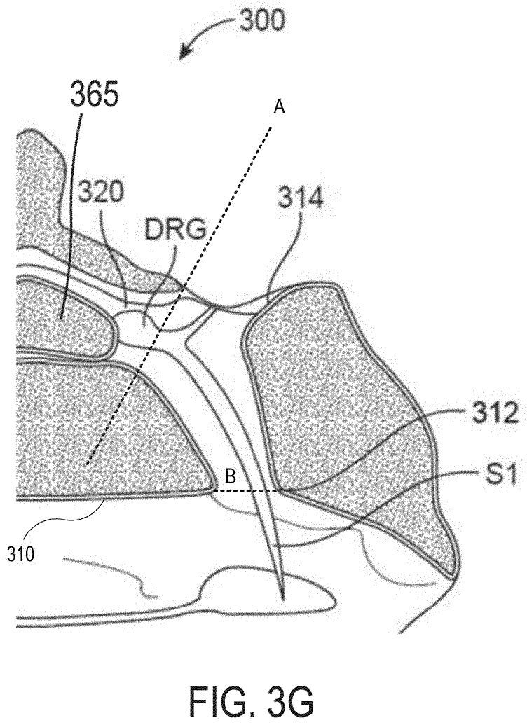

FIG. 3A illustrates a superior or coronal cross section of a sacrum 300 of a patient, through the S1 foramina of the sacral vertebral body. As shown here, the sacrum 300 includes a pair of S1 anterior (pelvic) foramen 312 and a pair of S1 posterior (dorsal) foramen 314. The S1 anterior foramen 312 are located at an anterior border or anterior (pelvic) surface 310 of the sacrum 300. In some patients, and as illustrated in FIG. 3A, the dorsal root ganglion DRG may be located within a spinal canal 320 of the sacrum 300. In some patients, the dorsal root ganglion DRG may be located in the vertebral body, and outside of the spinal canal (e.g. closer to the anterior foramen 312). If the DRG is located very closely to the entrance of the anterior foramen 312, a portion of the electrode region may be near the DRG during administration of the treatment. The treatment typically involves stimulating the nerve root at a target or treatment location that is distal to the DRG, as discussed elsewhere herein. In exemplary embodiments, a stimulation or modulation treatment is delivered by the treatment device 350 to a stimulation treatment location T that is outside of the spinal canal. Typically, the treatment is delivered by an electrode region 352 of the device 350. As discussed elsewhere herein, a surgeon or physician can use fluoroscopy to assist in guiding the electrode region of the device to and/or toward the desired treatment location. As shown here, the anterior or distal electrode of the device 350 is at or anterior to the anterior surface 310 of sacrum or sacral vertebral body 300, and the device 350 is generally positioned lateral to the thecal sac 360 which at this level (S1) contains the cauda equina 365. In some cases, the treatment device 350 can be anchored within the patient so that tines of the device 350 are disposed dorsal to the spinal canal 320. In some cases, the treatment device 350 can be anchored within the patient so that tines of the device 350 are disposed dorsal to a posterior border 315 of the bony sacrum 300. In some cases, a distal portion of the electrode region 352 is disposed anterior to an anterior border or surface 310 of the sacral vertebral body 300. In some cases, a distal portion of the electrode region 352 is disposed at the anterior border of the sacral vertebral body. In some cases, a distal portion of the electrode region 352 is disposed dorsal to the anterior border of the sacral vertebral body (e.g. within the spinal canal). In some cases, electrical stimulation is delivered by an electrode region 352 of the device at a treatment location that is located on the S1 nerve root anterior to the anterior border of the sacral vertebral body. In some cases, electrical stimulation is delivered by an electrode region 352 of the device at a treatment location that is located on the S1 nerve root at the anterior border 310 of the sacral vertebral body 300. In some cases, electrical stimulation is delivered by an electrode region 352 of the device at a treatment location that is located on the S1 nerve root dorsal to the anterior border 310 of the sacral vertebral body 300 (e.g. within the spinal canal).

FIG. 3B shows a lateral cross section view of a sacral vertebral body 300 of a patient. As shown here, the spinal canal 320 extends along a length 320L downward through the sacrum 300 to a location or boundary 340 that is between the S2 foramen and the S3 foramen. Thereafter, the spinal canal 320 transitions to the sacral canal 330. The sacral canal is a continuation of the spinal canal and extends downwardly along a length 330L throughout the lower part of the sacrum. Both the spinal canal 320 and the sacral canal 330 operate to lodge the sacral nerves, via the anterior and posterior sacral foramina. Typically, the spinal canal 320 lodges the S1 and S2 sacral nerves, and the sacral canal 330 lodges the S3 and S4 sacral nerves. The thecal or dural sac 360 extends downwardly through the spinal canal 320, and terminates at the boundary 340 that delineates the spinal canal 320 and sacral canal 330 (e.g. termination of the theca of the dura mater).

As shown in FIG. 3B, a sacrum 300 includes four posterior sacral foramina (e.g. S1 posterior sacral foramen S1P, S2 posterior sacral foramen S2P, S3 posterior sacral foramen S3P, and S4 posterior sacral foramen S4P). Typically, the posterior sacral foramina are smaller in size than the corresponding anterior sacral foramina The posterior sacral foramina give exit to the posterior divisions of the sacral nerves and the anterior sacral foramina give exit to the anterior divisions of the sacral nerves.

The distances between corresponding posterior and anterior sacral foramina vary. For example, the distance between the S1 posterior and anterior sacral foramina (S1P-S1A) is greater than the distance between the S2 posterior and anterior sacral foramina (S2P-S2A), which in turn is greater than the distance between the S3 posterior and anterior sacral foramina (S3P-S3A), which in turn is greater than the distance between the S4 posterior and anterior sacral foramina (S4P-S4A).

As discussed elsewhere herein, a treatment device can be anchored within a patient, such that a tined region of the treatment device is disposed dorsal or posterior to the spinal canal 320 (e.g. dorsal or posterior to the S1 posterior sacral foramen S1P). The treatment device can also be anchored such that a spacer region of the treatment device is disposed within the spinal canal 320 and lateral to the thecal sac 360 (e.g. between the S1 posterior sacral foramen S1P and the S1 anterior sacral foramen S1A). The treatment device can also be anchored such that a proximal portion of an electrode region is disposed within the spinal canal 320, and a distal portion of the electrode region is disposed anterior to an anterior border of the sacrum 300 (e.g. anterior to the S1 anterior sacral foramen S1A). The length of the spacer region is sufficiently long, so as to provide adequate distance between the treatment location and the tined region of the treatment device. For example, the spacer region has sufficient length, so that the tines are posterior to the thecal sac 360, and therefore do not damage or otherwise impinge upon the thecal sac 360. Typically, the tines engage or are anchored in muscle, fascia, and/or other tissue that is located posterior to the S1 posterior sacral foramen S1P. The thecal sac 360 contains fluid in which the spinal cord is situated. Because the thecal sac 360 does not extend downward past the S3 sacral foramina, surgical procedures performed through the S3 posterior sacral foramen S3P and/or the S3 anterior sacral foramen S3A do not present such a risk of damaging the thecal sac 360, as compared with surgical procedures that are performed through the S1 posterior sacral foramen S1P and/or the S1 anterior sacral foramen S1A. Hence, a treatment device configured for use through the S1 posterior sacral foramen S1P and/or the S1 anterior sacral foramen S1A will have the requisite dimensions and features, so as to not damage or unnecessarily impinge upon the thecal sac 360. According to some embodiments of the present invention, the distance between a treatment location (e.g. treatment location T depicted in FIG. 2A) and the thecal sac 360 is about 0.5 mm According to some embodiments of the present invention, exemplary method treatments include placing a nerve stimulation device through or along an insertion path that passes lateral to or outside the thecal sac 360, through the spinal canal 320, and into and through the S1 anterior sacral foramen of the sacral vertebral body.

Embodiments of the present invention encompass the use of a variety of techniques and devices for delivering treatment to a location on a spinal nerve root of an S1 nerve of a patient, and/or to other selected treatment location sites. For example, as illustrated in FIG. 3C, an electrode or other treatment device 300C can be advanced along a direction indicated by arrow A, through the sacral hiatus 310C, into the sacral canal 320C, and toward the dural sac termination 332C of the dural sac 330C (which may also be referred to as the thecal sac). Hence, whereas FIG. 3A depicts a posterior approach to reaching the treatment site, FIG. 3C depicts a caudal approach. FIG. 3D depicts the location of the apex 340C of the sacral hiatus from a side view, and FIG. 3E depicts the location of the apex 340C of the sacral hiatus from a posterior view. As shown here, the sacral hiatus 310C is positioned between two bony extensions 342C, 344C toward the bottom of the sacrum 305C. Typically, the sacral hiatus 310C and the apex 340C of the sacral hiatus are positioned toward the midline of the sacrum 305C.

Accordingly, FIGS. 3C to 3E illustrate aspects of an embodiment that involves advancing a lead or electrode (or other feature of a treatment device from a caudal to cephalic approach into the epidural space 315C of the sacral canal 320C, with access from the sacrococcygeal hiatus 310C at the apex 340C which is most commonly located at the level of S4, followed by S3 and S5, and sometimes as high as S1-S2.

The lead or electrode 300C can be advanced lateral to the dural sac 330C, which usually terminates between S1 and S2 vertebra, with the majority at S2, towards the S1 foramen in proximity to the S1 spinal nerve root. From there, it is possible to deliver an electrical stimulation treatment from the electrode region of the treatment device to a treatment location of the patient, as described elsewhere herein. For example, the treatment location can be disposed on a spinal nerve root of an S1 nerve of the patient. The treatment location can also be disposed distal to a dorsal root ganglion of the S1 nerve. Relatedly, the treatment location can be distal to a dorsal ramus of the S1 nerve. Similarly, the treatment location can be adjacent to an entry zone of a gray rami communicans of the S1 nerve.

During a treatment procedure, a surgeon can locate a landmark or anatomical feature on the patient's skin (e.g. on the patient's back), pierce the skin (e.g. with a needle), and advance the treatment device through the canal at a location where there is no dural sac, going up the sacral hiatus. Such techniques may involve the use of x-ray or fluoroscopy guidance, as described elsewhere herein, for example to achieve the desired placement of the device and delivery of the stimulation energy. In some cases, the sacral hiatus may be around the S3 or S4 level. In some cases, the sacral hiatus may be a certain number (e.g. 2) of levels below of where the stimulation target level. The surgeon can advance the device up the canal, and slightly lateral toward the foramen, where the nerve is going through. Such techniques can help avoid penetrating the dural sac (which may terminate at around the S1/S2 level. Hence, instead of coming from a transverse plane (posterior to anterior) such as that depicted in FIG. 3A, it is possible to advance the device from the bottom and in an upward direction.

As discussed elsewhere herein, embodiments of the present invention may include delivering an electrical stimulation treatment to a treatment location that is distal to a dorsal root ganglion of the S1 nerve. In some cases, embodiments may encompass methods and devices for delivering an electrical stimulation treatment that is at or near a dorsal root ganglion (DRG) of the S1 nerve, or at or near another dorsal root ganglion of the patient.

In some patients, with regard to the S1 DRG location, the S1 DRG may not be inside of the foramen but rather in the canal or at the junction of the canal, and therefore having a lead at the lateral portion exiting the foramen will not be stimulating at the DRG itself.

In some patients, the S1 DRG may be located in the intraforaminal (within the foramen) region. In some patients, the S1 DRG may be located in the intracanalar (inside the spinal canal medial to the foramen) region. In some cases, the S1 DRG may be located on or near the outer aspect of the foramen. In some embodiments, the S1 DRG may be located at the sacral border. In some cases, the S1 DRG may be posterior (e.g. inside the canal). In some embodiments, the S1 DRG may be anterior to the sacral border (e.g. extraforaminal) Embodiments of the present invention encompass devices and methods for delivering an electrical stimulation treatment to a treatment location on the spinal nerve root of an S1 nerve (or other nerve) of the patient, regardless of the location of the S1 DRG (or other DRG). Also, embodiments of the present invention encompass devices and methods for delivering an electrical stimulation treatment to a treatment location that is at or near the S1 DRG (or other DRG), regardless of the location of the S1 DRG (or other DRG). Often, embodiments involve stimulating at a location that is anterior to an anterior border of the sacral vertebral body, for example as depicted in FIG. 3A.

FIG. 3F depicts aspects of a caudal to cephalic approach. As shown here, a treatment device 300F can be advanced upwardly along the spinal cord 302F in the direction indicated by arrow F, and to a treatment location (*) that is extraforaminal and distal to a dorsal root ganglion DRG-1. As shown here, an intraspinal or intracanalar region can be defined between lines A1 and A2 which align medial borders of the pedicles P. Lines B1 and B2 can align the centers of the pedicles P, and an intraforaminal region can be between line A1 and line B1 and between line A2 and B2. An extraforaminal region can be located lateral to line B1 or B2. In this image, the lead turns at a curve or angle, goes through the intravertebral foramen. Embodiments of the present invention also encompass devices and techniques that involve directing the lead through the hiatus and placing the lead at the opening of the foramen from within the sacral canal in proximity to the sacral nerve as it approaches to exit the foramen. Relatedly, in some embodiments, the treatment location and corresponding patient anatomy (e.g. at spinal nerve root of an S1 nerve) may be disposed in the intraspinal or intracanalar region shown in FIG. 3F. Likewise, in some embodiments, the treatment location and corresponding patient anatomy (e.g. at spinal nerve root of an S1 nerve) may be disposed in the intraforaminal region shown in FIG. 3F. In some embodiments, the treatment location and corresponding patient anatomy (e.g. at spinal nerve root of an S1 nerve) may be disposed in the extraforaminal region shown in FIG. 3F.

FIG. 3G (which is based on FIG. 3A) depicts aspects of a posterior to anterior approach. As shown here, an intraspinal or intracanalar region can be disposed medial to dashed line A, an intraforaminal region can be disposed between dashed lines A and B, and an extraforaminal region can be disposed anterior to dashed line B. Hence, in contrast to the image provided in FIG. 3F where the regions can be defined relative to the pedicles P (i.e. in a lumbar or vertebral view), in FIG. 3G the regions can be defined relative to the posterior foramen 314 and the anterior foramen 312 (i.e. in a sacrum view). In some embodiments, the treatment location and corresponding patient anatomy (e.g. at spinal nerve root of an S1 nerve) may be disposed in the intraspinal or intracanalar region shown in FIG. 3G. Likewise, in some embodiments, the treatment location and corresponding patient anatomy (e.g. at spinal nerve root of an S1 nerve) may be disposed in the intraforaminal region shown in FIG. 3G. In some embodiments, the treatment location and corresponding patient anatomy (e.g. at spinal nerve root of an S1 nerve) may be disposed in the extraforaminal region shown in FIG. 3G.

Aspects of any of the S1 spinal nerve root and/or S1 DRG stimulation treatment methods or devices disclosed herein can also be used at other spinal levels to modulate sympathetic nervous system activity for other intended purposes related to sympathetic nervous system function. For example, embodiments encompass stimulation of any spinal nerve root upstream from the sympathetic chain ganglion of interest rather than stimulation of the DRG. In some cases, stimulation may involve stimulation of the DRG at a level that is different from the S1 level. In some cases, embodiments encompass stimulation of one or more of any combination of cervical, thoracic, lumbar, sacral, and/or coccygeal spinal nerves and/or nerve roots. In some cases, embodiments encompass stimulation of one or more of any combination of cervical, thoracic, lumbar, sacral, and/or coccygeal dorsal root ganglions.

According to some embodiments, exemplary methods can include advancing a treatment device along an insertion path into the patient, where the treatment device includes an electrode region, a spacer region located proximal to the electrode region, and a tined region located proximal to the spacer region, and where the insertion path is disposed lateral to a thecal sac, through a spinal canal, and into and through an S1 foramen of a sacral vertebral body of the patient.

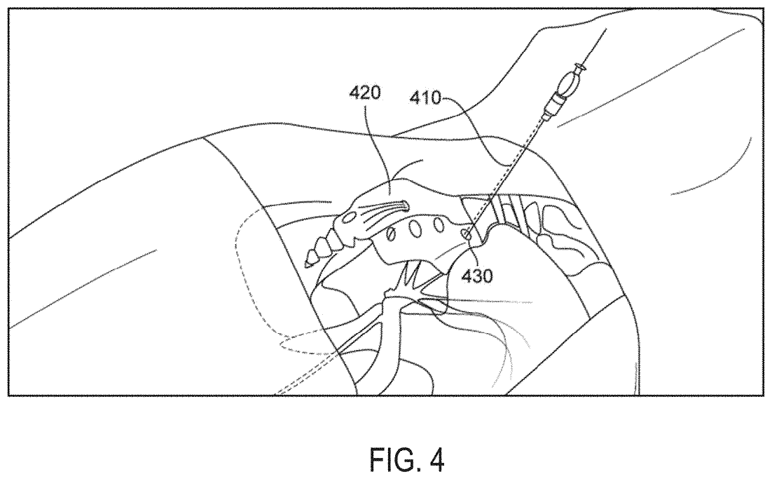

FIG. 4 depicts insertion of device along an insertion path 410, posteriorly through the sacrum 420 of a patient. In some surgical procedures, the surgeon locates the S1 posterior foramen 430 and inserts a tined lead. The tines deploy as a sheath of the device is removed. As discussed elsewhere herein, the treatment device can include an electrode region, a spacer region located proximal to the electrode region, and a tined region located proximal to the spacer region. The insertion path 410 can be disposed lateral to a thecal sac, through a spinal canal, and into and through an S1 anterior sacral foramen of the sacrum. The surgeon can operate the treatment device to administer selective electrical stimulation of a spinal nerve root of an S1 nerve, at a treatment location that is distal to a dorsal root ganglion (DRG) of the S1 nerve, so as to activate small fibers of the nerve. As noted elsewhere herein, such procedures can operate to modulate the afferent visceral input and the postganglionic sympathetic neurons at a treatment location on the S1 spinal nerve root NR, which is adjacent to an entry zone of a gray rami communicans of the S1 nerve. In this way, afferent visceral input (e.g. via visceral afferent sensory nerve fibers) and the postganglionic sympathetic neurons (e.g. efferent motor nerve fibers) can be modulated at the proximal S1 spinal nerve root.

Fluoroscopic guidance can be used by the physician (e.g. vascular surgeon) to facilitate the procedure. In some instances, a physician can align a fluoroscopic unit so as to visualize the S1 foramen on the desired side of treatment. A finder needle can be used to access the posterior S1 foramen and outline a desired path. A 14 g Tuohy needle can then follow the course of the finder needle and access the posterior S1 foramen. At that point the stylet can be removed from the Tuohy needle. A guide wire can then be passed through the Touhy needle in the lateral fluoroscopic view into the S1 anterior foramen. The guidewire can then be removed. The lead and introducer sheath can be passed into the Touhy needle with the final position of the lead to be with one contact anterior to the anterior vertebral body (e.g. anterior to the anterior S1 foramen) and the other contacts within the sacrum (e.g. posterior to the anterior S1 foramen and anterior to the posterior S1 foramen).

In some cases, a trial may be warranted. In such cases, a trial lead can be used. A trial lead may be similar to a treatment or implant lead as described elsewhere herein, except that a trial lead may not include tines. The trial lead may be anchored at the skin and may have an external generator system for the determined period of trial.

In an implant procedure, a physician can place an implant lead or nerve stimulation device in the patient. The implant lead typically includes tines. After placement of the lead in the final position, pressure can be maintained on the lead, and the sheath can then be slowly retracted. The procedure can be repeated on the contralateral side if the symptoms are bilateral. An implantable pulse generator, which may include or be part of a control device, can be implanted in the adjacent soft tissue of the lumbar spine. The implant pulse generator can be in operative communication with the implant lead.

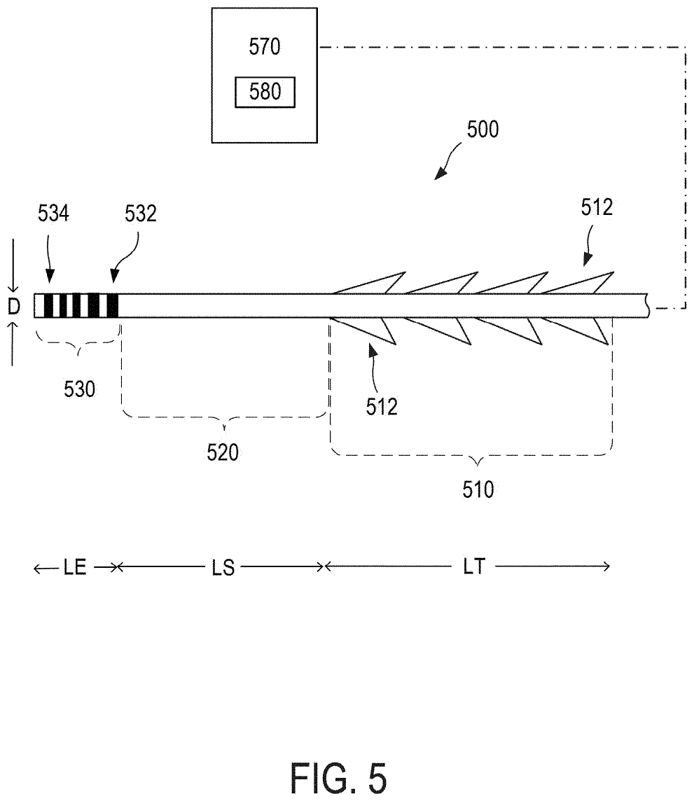

FIG. 5 depicts aspects of a nerve stimulation device or implant lead 500 according to embodiments of the present invention. As shown here, nerve stimulation device 500 includes a tined region 510, an electrode region 530, and a spacer region 520 located between the tined region 510 and the electrode region 530. The electrode region 530 is distal to the spacer region 520, and has a proximal portion or end 532 and a distal portion or end 534. The electrode region 530 has a diameter D. In some embodiments, the diameter D can have a value within a range from about 0.8 mm to about 1.4 mm In some embodiments, diameter D has a value of about 0.9 mm

The spacer region 520 is distal to the tined region 510. The spacer region 520 has a length LS. In some embodiments, the length LS of the spacer region 520 has a value within a range from about 15 mm to about 25 mm In some embodiments, the length LS of the spacer region 520 has a value within a range from about 22 mm to about 24 mm In some embodiments, length LS of the spacer region 520 has a value of about 20 mm The length LS of the spacer region can be selected so that when the device 500 is implanted in the patient, the tines 512 of the tined region 510 do not irritate or damage tissue that may be anterior to the posterior S1 sacral foramen. For example, the tines will not damage portions of the S1 sacral nerve, such as the nerve root, or the thecal (dural) sac.

In exemplary embodiments, the length LS is selected to accommodate the diameter or depth of the spinal canal. For example, in many patients, the diameter or depth of the spinal canal at the S1 level (e.g. distance between posterior S1 sacral foramen and anterior S1 sacral foramen) can be about 20 mm In some patients, the diameter or depth of the spinal canal at the S1 level can have a value between about 15 mm and about 25 mm

In contrast, the diameter or depth of the sacral canal at the S3 level (e.g. distance between posterior S3 sacral foramen and anterior S3 sacral foramen) can be about 9 mm Hence, it can be seen that the diameter or depth of the spinal canal at the S1 level is significantly greater than the diameter or depth of the sacral canal at the S3 level.

In some embodiments, the length LS is selected so that the activating electrode or electrodes of the electrode region 530 (e.g. at distal portion 534) are positioned significantly anterior to the dorsal root ganglion when the device is implanted in the patient. The electrode region 530 has a length LE. In some embodiments, the length LE of the electrode region 530 is about 20 mm The tined region 510 has a length LT. In some embodiments, the length LT of the tined region 510 is about 20 mm

As discussed elsewhere herein, a treatment method can include anchoring a treatment device 500 within the patient, such that the tined region 510 is disposed dorsal to the spinal canal, the spacer region 520 is disposed within the spinal canal and lateral to the thecal sac, the proximal portion 532 of the electrode region 532 is disposed within the spinal canal, and the distal portion 534 of the electrode region 530 is disposed anterior to an anterior border of the sacral vertebral body of the patient. In some cases, one or more tines are embedded in the thoracolumbar fascia.

In some embodiments, when positioning the nerve stimulation device 500, the tined region 510 is placed in a position dorsal or posterior to the spinal canal. The spacer region 520 can be placed in a position within the spinal canal and lateral to the thecal sac. The proximal end 532 of the electrode region 530 can be placed in a position within the spinal canal. The distal end 534 of the electrode region 530 can be placed in a position that is anterior to the anterior border of the sacral vertebral body.

Placement of an implant lead can be geared toward placement through the sacral foramen into the nerve root at the sacral plexus, with the electrical contacts anterior (outside) to the spinal canal. A lead may have visible and/or radiopaque markers that can be observed during the lead implantation procedure. In some cases, a lead or treatment device can include a plurality (e.g. 4 or 5) of electrodes and a plurality of tines (e.g. 4 or 5 tines). In use, the tines can operate to mitigate lead migration with measurements between the anchoring tines and the electrodes corresponding to anatomical dimensions at the S1 spinal canal and foramen.

Embodiments of the present invention encompass systems and methods that are configured to target the S1 spinal nerve (e.g. at a treatment location T as depicted in FIG. 2B) to improve blood flow in peripheral vascular disease and relieve associated ischemic pain. In doing so, the leads or treatment devices placed in the sacral foramen used at the S1 nerve root can have specific spacing measurements that correspond to the anatomic structures associated with the S1 nerve and S1 sacral foramina For example, a typical spinal canal diameter or thickness at the S1 foramina can be about 20 mm, and relatedly embodiments of the treatment device configuration can provide a distance between the anchoring tines and the electrodes that is about 20 mm, or greater, so that the tines do not deploy in the spinal canal (where they may otherwise damage or impinge upon the thecal sac), but rather deploy posterior to the spinal canal (e.g. posterior to the S1 posterior sacral foramen), in a way that fixes or anchors the electrodes relative to the S1 nerve root so that activated electrodes can modulate the S1 nerve root at the desired treatment location (e.g. at or near the entry zone of the gray rami communicans). This approach avoids engagement between the tines and the thecal sac. The thecal sac contains cerebrospinal fluid, spinal cord, and nerve roots, and ends at the S1/S2 level. By providing adequate distancing between the tines and the thecal sac, it is possible to avoid catastrophic complications which may otherwise occur if the tines engaged or impinged upon the thecal sac, because if there is ever a need for the lead or treatment device to be removed from the patient, or if there is traction placed on the lead or treatment device, engagement between the tines and the thecal sac could result in a tearing of the thecal sac thereby leading to a cascade of events which could require spinal surgery to repair this tear.

In exemplary embodiments, a treatment device 500 can include or be in operative association with a control unit 570. In some embodiments the control unit 570 may include or be in operative association with a user interface 580. In some cases, the control unit 570 can be an implantable pulse generator device.

The implantable pulse generator device can allow for small increment adjustments in the amplitude parameters (e.g. smaller than 0.1 mA increments) and have a stimulation frequency range starting at 0.5 Hz up to over 100 Hz. Very low frequency stimulation as low as 0.5 Hz can lead to inhibition of modulated neural output. By providing techniques that involve the ability to tune the electrical energy further with smaller amplitude increments and at lower frequencies, it is possible to achieve efficacies that heretofore have not been obtained.

Sensory afferent and sympathetic nervous system modulation can reduce vasoconstriction in distal peripheral arterial blood vessels and rapidly improve blood flow and perfusion to ischemic tissue. Furthermore, prolonged chronic inflammation resulting from PVD or other disorders like DPN can be reduced using such techniques.

Embodiments of the present invention can be used for patients with peripheral vascular disease causing significant lower extremity limb ischemia where S1 spinal nerve root stimulation will improve distal limb perfusion. In some embodiments, placement of leads at the S1 nerve root/sacral plexus can be facilitated using fluoroscopic guidance. The therapeutic outcome can be an increase in blood flow and tissue perfusion with concomitant pain relief secondary to modulation of sensory afferent input and sympathetic nerve output and reduced ischemia.

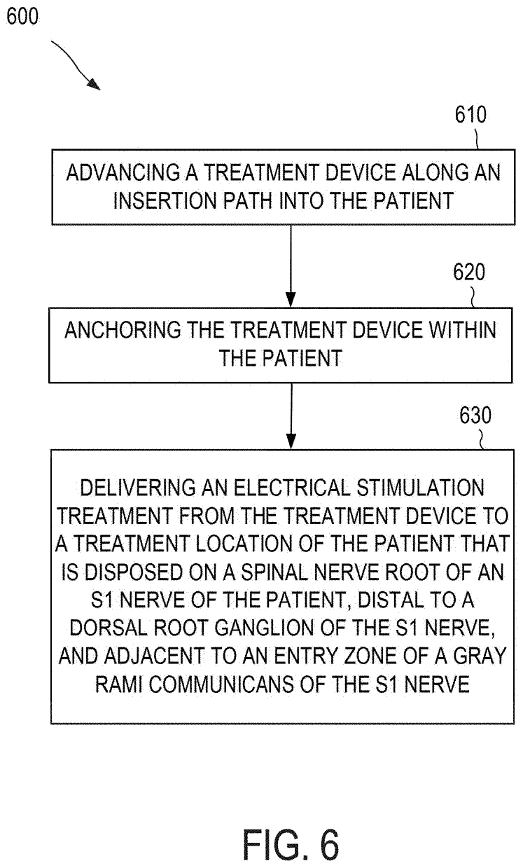

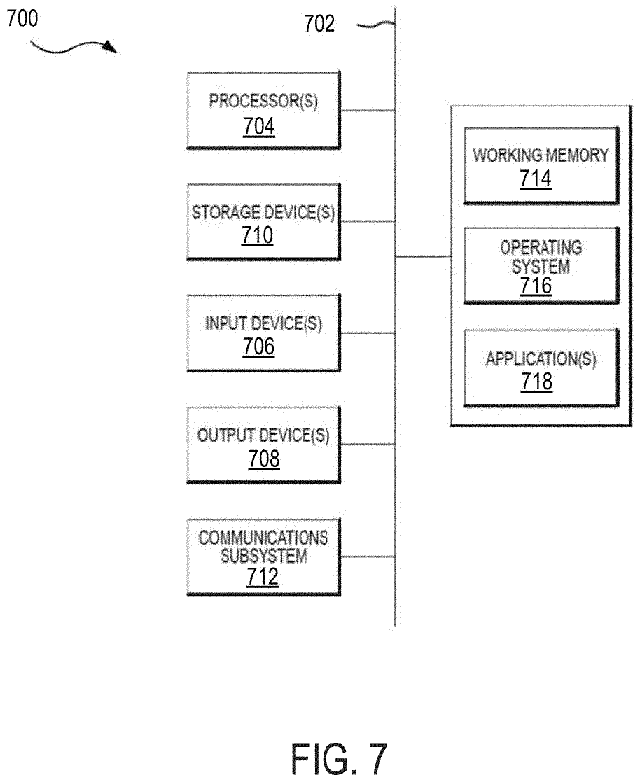

The control unit 570 can include or be in operative association with one or more processors (e.g. such as processor(s) 704 depicted in FIG. 7) configured with instructions for performing one or more method steps (e.g. such as method step 630 as depicted in FIG. 6), and operations as described elsewhere herein. Similarly, the control unit 570 may include or be in connectivity with any other component of a computer system (e.g. such as computer system 700 depicted in FIG. 7).

The control unit 570 can include or be in operative association with a power source. In some cases, the power source can be an internal battery (e.g. internal to the control unit). In some cases, the power source can be an external battery (e.g. external to the control unit). Relatedly, in some instances, the control unit 570 can include or be in operative association with an implantable pulse generator with an internal battery. In some instances, the control unit 570 can be external to the patient's body. In some embodiments, a power source in operative connectivity with a control unit 570 and/or a device 500 or lead or electrode can encompass any power source, regardless of whether the power source is internal or external to the control unit 570, and regardless of whether the power source is operated when inside (e.g. implanted) or outside (e.g. wearable or attachable) of the patient's body. Hence, embodiments of the present invention encompass both internal and external power source (e.g. battery) designs for delivering electricity or power, for example to a pulse generator, such as an implantable pulse generator, to power the leads or other aspects of the device. Hence, any permutation of a control unit (internal or external) in combination with a power source (internal or external) is encompassed by embodiments of the present invention. In some embodiments, a power source can be any type of power source, including without limitation a radiofrequency power source, a battery power source (including a rechargeable or a single use battery power source), a battery-free power source, and the like. In some cases, a stimulation system may include a pulse generator that is implanted in the patient and a power source that is external to the patient (e.g. wearable power source). The power source and pulse generator can be in operative communication via a wired or wireless connection. In some cases, the connectivity between the power source and the pulse generator can involve the use of an antenna and/or receiver.

FIG. 6 depicts aspects of an exemplary method 600 for treating a patient, according to embodiments of the present invention. In some cases, the patient may be presenting with peripheral vascular disease. As shown here, method 600 includes advancing a treatment device along an insertion path into the patient, as indicated by step 610. In some cases, the treatment device includes an electrode region, a spacer region located proximal to the electrode region, and a tined region located proximal to the spacer region. In some cases, the insertion path is disposed lateral to a thecal sac, through a spinal canal, and into and through an S1 foramen of a sacral vertebral body of the patient. Method 600 may also include anchoring the treatment device within the patient, as indicated by step 620. In some cases, the tined region is disposed dorsal to the spinal canal, the spacer region is disposed within the spinal canal and lateral to the thecal sac, a proximal portion of the electrode region is disposed within the spinal canal, and a distal portion of the electrode region is disposed anterior to an anterior border of the sacral vertebral body of the patient. Method 600 may also include delivering an electrical stimulation treatment from the electrode region of the treatment device to a treatment location of the patient, as indicated by step 630. The treatment location may be disposed on a spinal nerve root of an S1 nerve of the patient, distal to a dorsal root ganglion of the S1 nerve, and adjacent to an entry zone of a gray rami communicans of the S1 nerve.

FIG. 7 depicts aspects of an exemplary computer system or device 700 configured for use with any of the treatment devices or methods disclosed herein, according to embodiments of the present invention. An example of a computer system or device 700 may include an enterprise server, blade server, desktop computer, laptop computer, tablet computer, personal data assistant, smartphone, any combination thereof, and/or any other type of machine configured for performing calculations. Any computing devices encompassed by embodiments of the present invention may be wholly or at least partially configured to exhibit features similar to the computer system 700.

The computer system 700 of FIG. 7 is shown comprising hardware elements that may be electrically coupled via a bus 702 (or may otherwise be in communication, as appropriate). The hardware elements may include a processing unit with one or more processors 704, including without limitation one or more general-purpose processors and/or one or more special-purpose processors (such as digital signal processing chips, graphics acceleration processors, and/or the like); one or more input devices 706, which may include without limitation a remote control, a mouse, a keyboard, a keypad, a touchscreen, and/or the like; and one or more output devices 708, which may include without limitation a presentation device (e.g., controller screen, display screen), a printer, and/or the like.

The computer system 700 may further include (and/or be in communication with) one or more non-transitory storage devices 710, which may comprise, without limitation, local and/or network accessible storage, and/or may include, without limitation, a disk drive, a drive array, an optical storage device, a solid-state storage device, such as a random access memory, and/or a read-only memory, which may be programmable, flash-updateable, and/or the like. Such storage devices may be configured to implement any appropriate data stores, including without limitation, various file systems, database structures, and/or the like.

The computer system 700 can also include a communications subsystem 712, which may include without limitation a modem, a network card (wireless and/or wired), an infrared communication device, a wireless communication device and/or a chipset such as a Bluetooth device, 802.11 device, WiFi device, WiMax device, cellular communication facilities such as GSM (Global System for Mobile Communications), W-CDMA (Wideband Code Division Multiple Access), LTE (Long Term Evolution), and the like. The communications subsystem 712 may permit data to be exchanged with a network (such as the network described below, to name one example), other computer systems, controllers, and/or any other devices described herein. In many embodiments, the computer system 700 can further comprise a working memory 714, which may include a random access memory and/or a read-only memory device, as described above.

The computer system 700 also can comprise software elements, shown as being currently located within the working memory 714, including an operating system 1716, device drivers, executable libraries, and/or other code, such as one or more application programs 718, which may comprise computer programs provided by various embodiments, and/or may be designed to implement methods, and/or configure systems, provided by other embodiments, as described herein. By way of example, one or more procedures described with respect to the method(s) discussed herein, and/or system components might be implemented as code and/or instructions executable by a computer (and/or a processor within a computer); in an aspect, then, such code and/or instructions may be used to configure and/or adapt a general purpose computer (or other device) to perform one or more operations in accordance with the described methods.

A set of these instructions and/or code can be stored on a non-transitory computer-readable storage medium, such as the storage device(s) 710 described above. In some cases, the storage medium might be incorporated within a computer system, such as computer system 700. In other embodiments, the storage medium might be separate from a computer system (e.g., a removable medium, such as flash memory), and/or provided in an installation package, such that the storage medium may be used to program, configure, and/or adapt a general purpose computer with the instructions/code stored thereon. These instructions might take the form of executable code, which is executable by the computer system 700 and/or might take the form of source and/or installable code, which, upon compilation and/or installation on the computer system 700 (e.g., using any of a variety of generally available compilers, installation programs, compression/decompression utilities, and the like), then takes the form of executable code.

It is apparent that substantial variations may be made in accordance with specific requirements. For example, customized hardware might also be used, and/or particular elements might be implemented in hardware, software (including portable software, such as applets, and the like), or both. Further, connection to other computing devices such as network input/output devices may be employed.

As mentioned elsewhere herein, in one aspect, some embodiments may employ a computer system (such as the computer system 700) to perform methods in accordance with various embodiments of the disclosure. According to a set of embodiments, some or all of the procedures of such methods are performed by the computer system 700 in response to processor 704 executing one or more sequences of one or more instructions (which might be incorporated into the operating system 716 and/or other code, such as an application program 718) contained in the working memory 714. Such instructions may be read into the working memory 714 from another computer-readable medium, such as one or more of the storage device(s) 710. Merely by way of example, execution of the sequences of instructions contained in the working memory 714 may cause the processor(s) 1704 to perform one or more procedures of the methods described herein.

The terms “machine-readable medium” and “computer-readable medium,” as used herein, can refer to any non-transitory medium that participates in providing data that causes a machine to operate in a specific fashion. In an embodiment implemented using the computer system 700, various computer-readable media might be involved in providing instructions/code to processor(s) 704 for execution and/or might be used to store and/or carry such instructions/code. In many implementations, a computer-readable medium is a physical and/or tangible storage medium. Such a medium may take the form of a non-volatile media or volatile media. Non-volatile media may include, for example, optical and/or magnetic disks, such as the storage device(s) 710. Volatile media may include, without limitation, dynamic memory, such as the working memory 714.

Exemplary forms of physical and/or tangible computer-readable media may include a floppy disk, a flexible disk, hard disk, magnetic tape, or any other magnetic medium, a compact disc, any other optical medium, ROM, RAM, and the like, any other memory chip or cartridge, or any other medium from which a computer may read instructions and/or code. Various forms of computer-readable media may be involved in carrying one or more sequences of one or more instructions to the processor(s) 704 for execution. By way of example, the instructions may initially be carried on a magnetic disk and/or optical disc of a remote computer. A remote computer might load the instructions into its dynamic memory and send the instructions as signals over a transmission medium to be received and/or executed by the computer system 700.

The communications subsystem 712 (and/or components thereof) generally can receive signals, and the bus 702 then can carry the signals (and/or the data, instructions, and the like, carried by the signals) to the working memory 714, from which the processor(s) 704 retrieves and executes the instructions. The instructions received by the working memory 714 may optionally be stored on a non-transitory storage device 710 either before or after execution by the processor(s) 704.

It should further be understood that the components of computer system 700 can be distributed across a network. For example, some processing may be performed in one location using a first processor while other processing may be performed by another processor remote from the first processor. Other components of computer system 700 may be similarly distributed. As such, computer system 700 may be interpreted as a distributed computing system that performs processing in multiple locations. In some instances, computer system 700 may be interpreted as a single computing device, such as a distinct laptop, desktop computer, or the like, depending on the context.

A processor may be a hardware processor such as a central processing unit (CPU), a graphic processing unit (GPU), or a general-purpose processing unit. A processor can be any suitable integrated circuits, such as computing platforms or microprocessors, logic devices and the like. Although the disclosure is described with reference to a processor, other types of integrated circuits and logic devices are also applicable. The processors or machines may not be limited by the data operation capabilities. The processors or machines may perform 512 bit, 256 bit, 128 bit, 64 bit, 32 bit, or 16 bit data operations.

Each of the calculations or operations discussed herein may be performed using a computer or other processor having hardware, software, and/or firmware. The various method steps may be performed by modules, and the modules may comprise any of a wide variety of digital and/or analog data processing hardware and/or software arranged to perform the method steps described herein. The modules optionally comprising data processing hardware adapted to perform one or more of these steps by having appropriate machine programming code associated therewith, the modules for two or more steps (or portions of two or more steps) being integrated into a single processor board or separated into different processor boards in any of a wide variety of integrated and/or distributed processing architectures. These methods and systems will often employ a tangible media embodying machine-readable code with instructions for performing the method steps described herein. All features of the described systems are applicable to the described methods mutatis mutandis, and vice versa. Suitable tangible media may comprise a memory (including a volatile memory and/or a non-volatile memory), a storage media (such as a magnetic recording on a floppy disk, a hard disk, a tape, or the like; on an optical memory such as a CD, a CD-R/W, a CD-ROM, a DVD, or the like; or any other digital or analog storage media), or the like. While the exemplary embodiments have been described in some detail, by way of example and for clarity of understanding, those of skill in the art will recognize that a variety of modification, adaptations, and changes may be employed.

According to some embodiments, machine-readable code instructions for, and/or data generated or used by, treatment devices and/or computing devices (which may include smart phones or other mobile computing devices) can be stored on or executed by any of a variety of computing modalities, including without limitation personal computers, servers (e.g. hosted and/or privately owned servers) , internet connections, cloud hosts, cloud based storage, and the like.

Operational Modulation Parameters

As described elsewhere herein, a treatment device can include or be in operative association with a control unit. In some embodiments the control unit may include or be in operative association with a user interface. The control unit can include or be in operative association with one or more processors configured with instructions for performing one or more method steps (e.g. delivering modulation energy to a treatment location of a patient. A control unit may include or be in connectivity with any component of a computer system.

In some cases, a computer or control unit can be programmed or include instructions to deliver modulation energy having certain parameters. For example, modulation energy parameters may involve delivering electrical energy to the treatment location, where the electrical energy has a frequency having a value within a range from about 0.5 Hz to about 20 Hz. Such frequencies can be well suited for use in promoting pain inhibition. In some cases, the electrical energy can have a frequency having a value that is greater than about 20 Hz. Such frequencies may be useful for providing other treatment results to a patient.

In some cases, electrical energy can be delivered at a constant current (e.g. rather than at a constant voltage). In some cases, systems or methods can involve delivering electrical energy at current values that can be selected based on certain intervals. For example, energy can be delivered at a first current value, and thereafter delivered at a second current value that differs from the first current value by about 0.005 milliamps In some cases, energy can be delivered at a first current value, and thereafter delivered at a second current value that differs from the first current value by about 0.025 milliamps In some cases, a difference between an earlier electrical energy current level and a subsequent energy current level can have a value within a range from about 0.005 milliamps to about 0.025 milliamps

A control unit or implantable pulse generator device can allow for small increment adjustments in the amplitude parameters (e.g. current increments having a value within a range from about 0.005 mA to about 0.025 mA). A control unit or implantable pulse generator device can allow for the delivery of stimulation energy (e.g. via a treatment device) having a frequency value within a range from about 0.5 Hz to about 100 Hz. In some cases, a control unit or implantable pulse generator device can allow for the delivery of stimulation energy (e.g. via a treatment device) having a frequency value that is greater than 100 Hz. Delivery of very low frequency stimulation, for example as low as 0.5 Hz, can lead to inhibition of modulated neural output. By providing the ability to tune the electrical energy with small amplitude increments (e.g. current increments having a value within a range from about 0.005 mA to about 0.025 mA) and/or at low frequencies (a frequency having a value within a range from about 0.5 Hz to about 20 Hz) it is possible to achieve a desired effect.

According to some embodiments, by providing treatments having selected frequencies and/or current amplitudes, it is possible to activate certain nerve fibers and/or achieve certain results. For example, the delivery of low frequency energy (e.g. as low as 0.5 Hz) can operate to activate inhibition. As another example, the delivery of energy at low current increments (e.g. current increments having a value within a range from about 0.005 mA to about 0.025 mA) it is possible to finely tune and capture and/or activate one or more desired nerves without activating the nerves which cause paresthesia, or tingling, sensations. According to some embodiments, by providing treatments having selected frequencies and/or current amplitudes, it is possible to activate the sympathetic nerves, which are A delta and C fibers.

A constant current system can operate to deliver the same voltage across the electrodes, so if there is an impedance to delivery in one area it will not go through. In contrast, with position changes, a constant voltage system can tend to cause shock sensations.

Indications

Selective stimulation of the S1 spinal nerve root NR can operate to activate small fibers of the S1 nerve, including sympathetic nerves (e.g. POST-GSN) which can improve blood flow, relieve or reduce ischemic pain, and improve limb salvage. Small fibers (e.g. sensory nerves fibers) also innervate the skin of the extremity and thus stimulation can treat neuropathic pain.

Blood flow and pressure are controlled by a class of nerves referred to as sympathetic nerves, which are activated either directly by sensory nerves coming from the local blood vessel walls and surrounding tissue or indirectly by descending brain circuits that process the sensory nerve information first. In a patient at a normal healthy resting state, sympathetic nerves maintain a baseline tone in the blood vessels by constricting the muscles in the vessel walls (referred to as vasoconstriction) enough to maintain a level of resistance or pressure that ensures blood travels forward while also being able to overcome gravity and return to the heart. Increasing sympathetic nerve activity increases vasoconstriction which reduces blood flow or perfusion to surrounding tissues. Therefore, selectively applying electrical stimulation to modulate those sensory nerve fibers that signal the sympathetic nerves and the brain regions controlling them will have a greater impact on restoring blood flow to regions such as the distal legs and feet affected by peripheral vascular disease. It has been discovered that method embodiments disclosed herein can result in an improvement of such blood flow.

An exemplary location to selectively target these sensory nerve fibers associated with the sympathetic nerves is at the S1 spinal nerve root before entry into the spinal cord (e.g. adjacent to an entry zone of a gray rami communicans of the S1 nerve). Targeting the S1 sacral nerve root can be particularly effective for treating patients presenting with PVD, because PVD usually affects the distal lower extremities and feet most due to the greater distance from the heart.

In addition or instead of treating PVD, the application of a modulation treatment protocol to a location adjacent to an entry zone of a gray rami communicans of the S1 nerve can also operate as a treatment for patients presenting with diabetic neuropathy. Because the treatment location is at the spinal nerve root, it is possible to activate and/or finely tine the sympathetic nerve fibers, which can improve sympathetic blood supply, reduce or ameliorate neuropathic pain in the patient's feet, and treat diabetic neuropathy and other forms of neuropathy.

The administration of sympathetic nerve stimulation protocols as disclosed herein can operate to improve blood flow and tissue perfusion (e.g. in peripheral vascular disease), relieve ischemic pain, and improve limb salvage. In some embodiments, the administration of stimulation protocols as disclosed herein can operate to modulate sensory afferent input. The small fibers also innervate the skin of the extremity, and thus stimulation can treat neuropathic pain. In exemplary embodiments, the treatment outcome can include increases in blood flow and tissue perfusion with concomitant pain relief secondary to modulation of sensory afferent input and reduced ischemia.

According to some embodiments, visceral afferent and sympathetic nervous system modulation techniques as disclosed herein can operate to reduce vasoconstriction in distal peripheral arterial blood vessels and rapidly improve blood flow and perfusion to ischemic tissue. Furthermore, prolonged chronic inflammation resulting from PVD or other disorders like diabetic peripheral neuropathy (DPN) could be reduced.

System and method embodiments disclosed herein can be used for patients with peripheral vascular disease causing significant lower extremity limb ischemia where S1 nerve root stimulation will improve distal limb perfusion. In some cases, fluoroscopic guidance may be used to assist in placing leads at the S1 nerve root/sacral plexus. In some cases, the therapeutic outcome can include an increase in blood flow and tissue perfusion with concomitant pain relief secondary to modulation of sensory afferent input and sympathetic nerve output and reduced ischemia.

Blood flow and pressure are controlled by the sympathetic nervous system, which are small nerve fibers that innervate the vasculature and intrinsic organs of the body. These nerves are activated either directly by sensory nerves coming from the local blood vessel walls and surrounding tissue or indirectly by descending brain circuits that process the sensory input first. In the normal healthy resting state, sympathetic nerves maintain a baseline tone in the blood vessels by constricting the muscles in the vessel walls (referred to as vasoconstriction) enough to maintain a level of resistance or pressure that ensures blood travels forward while also being able to overcome gravity and return to the heart. Increasing sympathetic nerve activity increases vasoconstriction which reduces blood flow or perfusion to surrounding tissues. The ischemic environment, caused by interrupted blood flow, affects the supply of nutrients and elongates the inflammation period, inducing tissue degeneration due to accumulation of toxic metabolites and inflammatory molecules such as oxygen radicals and cytokines that are normally flushed out with sufficient blood flow. Therefore, selectively applying electrical stimulation to modulate sensory nerve fibers and the sympathetic nerves they signal can have a significant impact on restoring blood flow and reducing associated inflammation to the distal legs and feet affected by PVD.

Very low frequency stimulation, for example as low as 0.5 Hz, can lead to inhibition of modulated neural output, and by providing systems and methods that enable the tuning of the electrical energy with small amplitude increments (e.g. current increments having a value within a range from about 0.005 mA to about 0.025 mA), it is possible to provide efficient and effective treatments.

System and method embodiments disclosed herein are well suited for use by vascular surgeons, as they are the managers of patients with PVD. Stimulating the S1 nerve root using certain parameters, at a certain location in the spine will improve distal limb perfusion. Placing the lead at the S1 nerve root (e.g. adjacent to an entry zone of a gray rami communicans of the S1 nerve) can be facilitated with fluoroscopic guidance, for example using an X-ray machine that vascular surgeons routinely use for vascular interventions such as angioplasty. The treatment outcome includes increases in blood flow and tissue perfusion with concomitant pain relief secondary to modulation of sensory afferent input and reduced ischemia. Embodiments of the present invention can be used to modulate sympathetic nerve output to reduce vasoconstriction. Exemplary systems and methods can be configured for use in S1 sacral nerve root stimulation.

All publications, patents, patent applications, journal articles, books, technical references, and the like mentioned in this specification are herein incorporated by reference to the same extent as if each individual publication, patent, patent application, journal article, book, technical reference, or the like was specifically and individually indicated to be incorporated by reference.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating a patient presenting with peripheral vascular disease or diabetic peripheral neuropathy, the method comprising:
advancing a treatment device along an insertion path into the patient, wherein the treatment device comprises an electrode region, a spacer region located proximal to the electrode region, and a tined region located proximal to the spacer region, and wherein the insertion path is disposed lateral to a thecal sac, through a spinal canal, and into and through an S1 foramen of a sacral vertebral body of the patient;
anchoring the treatment device within the patient, such that the tined region is disposed dorsal to the spinal canal and dorsal to a posterior border of the sacral vertebral body of the patient, the spacer region is disposed within the spinal canal and lateral to the thecal sac, a proximal portion of the electrode region is disposed within the spinal canal, and a distal portion of the electrode region is disposed anterior to an anterior border of the sacral vertebral body of the patient; and
delivering an electrical stimulation treatment from the electrode region of the treatment device to a treatment location of the patient, wherein the treatment location is disposed on a spinal nerve root of an S1 nerve of the patient, distal to a dorsal root ganglion of the S1 nerve, distal to a dorsal ramus of the S1 nerve, and adjacent to an entry zone of a gray rami communicans of the S1 nerve, and wherein delivery of the electrical stimulation treatment operates to simultaneously modulate visceral afferent fibers and postganglionic sympathetic neurons at the treatment location.

2. The method according to claim 1, wherein the step of anchoring the treatment device within the patient comprises embedding one or more tines of the tined region of the treatment device in thoracolumbar fascia tissue of the patient.

3. The method according to claim 1, wherein the spacer region of the treatment device has a length of about 20 mm

4. The method according to claim 1, wherein the spacer region of the treatment device has a length with a value within a range from about 22 mm to about 24 mm

5. The method according to claim 1, wherein the spacer region of the treatment device has a length with a value within a range from about 15 mm to about 25 mm

6. The method according to claim 1, wherein the step of delivering the electrical stimulation treatment comprises delivering electrical energy with a frequency having a value within a range from about 0.5 Hz to about 20 Hz.

7. The method according to claim 1, wherein the step of delivering the electrical stimulation treatment comprises delivering electrical energy with a frequency having a value that is greater than 20 Hz.

8. The method according to claim 1, wherein the step of delivering the electrical stimulation treatment comprises delivering electrical energy with a frequency having a value that is greater than 100 Hz.

9. The method according to claim 1, wherein the step of delivering the electrical stimulation treatment comprises delivering electrical energy at a first current value and subsequently at a second current value, wherein the first current value and the second current value are separated by a current increment, and wherein the current increment has a value within a range from about 0.005 mA to about 0.025 mA.

10. The method according to claim 1, wherein the step of delivering the electrical stimulation treatment comprises delivering electrical energy at a constant current.

11. A system for treating a patient presenting with peripheral vascular disease, the system comprising:
a treatment device having an electrode region, a spacer region located proximal to the electrode region, and a tined region located proximal to the spacer region, wherein the treatment device is configured for anchoring within the patient, such that the tined region is disposed dorsal to an S1 posterior sacral foramen of the patient, the spacer region is disposed within the spinal canal and lateral to a thecal sac of the patient, a proximal portion of the electrode region is disposed within the spinal canal, a distal portion of the electrode region is disposed anterior to an S1 anterior sacral foramen corresponding to the S1 posterior sacral foramen, and an electrode of the electrode region is disposed at a spinal nerve root of an S1 nerve of the patient, distal to a dorsal root ganglion of the S1 nerve, distal to a dorsal ramus of the S1 nerve, and adjacent to an entry zone of a gray rami communicans of the S1 nerve;
a processor;
an electronic storage location operatively coupled with the processor; and
processor executable code stored on the electronic storage location and embodied in a tangible non-transitory computer readable medium,
wherein the processor executable code, when executed by the processor, causes the treatment device to deliver an electrical stimulation treatment from the electrode region of the treatment device to the patient, to simultaneously modulate visceral afferent fibers and postganglionic sympathetic neurons of the patient.

12. The system according to claim 11, wherein the spacer region of the treatment device has a length of about 20 mm

13. The system according to claim 1, wherein the spacer region of the treatment device has a length with a value within a range from about 22 mm to about 24 mm

14. The system according to claim 1, wherein the spacer region of the treatment device has a length with a value within a range from about 15 mm to about 25 mm

15. The system according to claim 1, wherein the electrical stimulation treatment comprises electrical energy with a frequency having a value within a range from about 0.5 Hz to about 20 Hz.

16. The system according to claim 1, wherein the electrical stimulation treatment comprises electrical energy with a frequency having a value that is greater than 20 Hz.

17. The system according to claim 1, wherein the electrical stimulation treatment comprises electrical energy with a frequency having a value that is greater than 100 Hz.

18. The system according to claim 1, wherein the electrical stimulation treatment comprises electrical energy provided at a first current value and subsequently at a second current value, wherein the first current value and the second current value are separated by a current increment, and wherein the current increment has a value within a range from about 0.005 mA to about 0.025 mA.

19. The system according to claim 1, wherein the electrical stimulation treatment comprises electrical energy provided at a constant current.

20. A method of stimulating a spinal nerve root of an S1 nerve of a patient, the method comprising:
advancing a treatment device along an insertion path into the patient, wherein the treatment device comprises an electrode region, a spacer region located proximal to the electrode region, and a tined region located proximal to the spacer region, and wherein the insertion path is disposed lateral to a thecal sac, through a spinal canal, and into and through an S1 foramen of a sacral vertebral body of the patient;
anchoring the treatment device within the patient, such that the tined region is disposed dorsal to the spinal canal, the spacer region is disposed lateral to the thecal sac, and an electrode of the electrode region is disposed to an anterior border of the sacral vertebral body of the patient; and
delivering an electrical stimulation treatment from the electrode of the treatment device to a treatment location of the patient, wherein the treatment location is disposed on a spinal nerve root of an S1 nerve of the patient, distal to a dorsal root ganglion of the S1 nerve, distal to a dorsal ramus of the S1 nerve, and adjacent to an entry zone of a gray rami communicans of the S1 nerve, and wherein delivery of the electrical stimulation treatment operates to simultaneously modulate visceral afferent fibers and postganglionic sympathetic neurons at the treatment location,
wherein the patient presents with a condition selected from the group consisting of peripheral vascular disease, diabetic peripheral neuropathy, complex regional pain syndrome, and Raynaud's syndrome.

21. A method of treating a patient, the method comprising:
advancing a treatment device along an insertion path into the patient, wherein the treatment device comprises an electrode region, and wherein the insertion path is disposed through a foramen of a vertebral body of the patient;
anchoring the treatment device within the patient, such that a distal portion of the electrode region is in an extraforaminal region; and
delivering an electrical stimulation treatment from the electrode region of the treatment device to a treatment location of the patient, wherein the treatment location is disposed on a spinal nerve root of a spinal nerve of the patient, and wherein delivery of the electrical stimulation treatment operates to simultaneously modulate visceral afferent fibers and postganglionic sympathetic neurons at the treatment location.
